(19) United States
(12) Patent Application Publication
(10) Pub. No.: US 2024/0041768 A1
(43) Pub. Date: Feb. 8, 2024

(54) MULTIFUNCTIONAL pO2/pH-SENSITIVE THERANOSTIC LIPOSOME NANOCARRIERS AND METHODS OF USING SAME

(71) Applicant: YALE UNIVERSITY, New Haven, CT (US)

(72) Inventors: Samuel Maritim, New Haven, CT (US); Ligia Boisserand, New Haven, CT (US); Fahmeed Hyder, New Haven, CT (US); Lauren Sansing, New Haven, CT (US)

(21) Appl. No.: 18/357,810

(22) Filed: Jul. 24, 2023

Related U.S. Application Data
(60) Provisional application No. 63/391,895, filed on Jul. 25, 2022.

(51) Int. Cl. A61K 9/127; A61K 45/06; A61K 31/7076; A61K 51/12

(52) U.S. Cl. CPC A61K 9/127; A61K 45/06; A61K 31/7076; A61K 51/1234; B82Y 5/00

(57) ABSTRACT

Provided herein are hypoxia/acidic targeting compounds formulated in lipid-containing nanoparticles (liposomes) containing a diagnostic and/or a therapeutic agent. These nanoparticles can penetrate the blood-brain barrier (BBB) and are useful in the treatment ischemic conditions, as well as systemic conditions with hypoxic environments, such as tumors.

### CROSS-REFERENCE TO RELATED APPLICATION

The present application claims priority under 35 U.S.C. § 119(e) to U.S. Provisional Patent Application No. 63/391,895, filed Jul. 25, 2022, the disclosure of which is incorporated herein by reference in its entirety.

### STATEMENT REGARDING FEDERALLY SPONSORED RESEARCH

This invention was made with government support under TR001864 awarded by the National Institutes of Health. The government has certain rights in the invention.

### BACKGROUND

Ischemia, a pathological condition in which tissue is deprived of adequate blood flow (and thus oxygen and glucose supply). During ischemic stroke, blood supply to the brain is severely disrupted (e.g., due to blood clots, cerebral thrombosis, or embolism) resulting in inadequate oxygen supply (hypoxia) to brain cells, glycolytic metabolism, oxidative energy failure, ionic imbalance and eventually cell death. Often, the ischemic microenvironment is characterized by acidic conditions (lower pH) compared to healthy well oxygenated tissue likely due to a combination of metabolic alterations that generate excess lactic acid (due to upregulation of glycolysis), poor perfusion and disturbance in clearance of the acidic metabolic waste. Hypoxia and low tissue pH are linked to most of the insidious hallmarks of ischemic pathologies including generation of free radicals, impaired protein synthesis, activation of cytokine and inflammatory pathways, membrane degradation, accumulation of intracellular calcium, cytotoxic edema, and cell death. Nevertheless, tissue acidity and hypoxia may be exploited to develop highly specific diagnostic and therapeutic agents that target ischemic tissues. For example, agents may be developed to bind and/or release drugs only to hypoxic and acidic tissue. Because conditions like hypoxia and low extracellular pH are rarely found in healthy brain tissue, agents that specifically target hypoxia and low pH are highly desirable. These targeted agents/nanocarriers would deliver high payloads of theranostic agents specifically to desired diseased tissue, minimize off-target toxicities, overcome natural barriers like the blood brain barrier, reduce dosage/amount of required drug and reduce cost. Furthermore, diagnostic and theranostic agents targeted to ischemic microenvironment may provide a means to assess the efficacy of therapeutic interventions e.g., through changes in lesion size, BBB permeability, changes in lesion microenvironment (pH, pO₂, lactate levels) and tracer uptake/accumulation. The present invention focuses on ischemic stroke, but the principles and inventions herein can also apply to other ischemic pathologies e.g., solid tumors, traumatic brain injury, heart disease, lung disorders, organ ischemia and rheumatoid arthritis.

Theranostic agents linked to ischemia-sensitive agents may achieve high target specificity, but are likely to still face challenges of solubility, clearance, short circulation time, degradation, permeability and subtherapeutic delivery. Recently, nanocarriers have been used to overcome most of these challenges. These are nanosized vehicles or particles whereby therapeutic and/or diagnostic agents may be incorporated, encapsulated within, or linked to the surface of the particle. Some of the nanocarriers that have been utilized in drug delivery include micelles, polymeric nanoparticles, protein nanocarriers, ferrites, quantum dots, organic nanotubes, dendrimers, solid lipid nanoparticles, nanostructured lipid carriers, and liposomes. Of these classes of nanocarriers, liposomes are the most successful in part because they are biocompatible, non-toxic, non-immunogenic, can be loaded with more than one type of cargo of varying solubilities and can be targeted e.g., to diseased tissue.

Liposomes are phospholipid bilayer vesicles with an internal aqueous core and a fatty lipid bilayer membrane. They may have one bilayer (unilamellar vesicles) or >1 concentric bilayers (multilamellar vesicles) and range from <50 nm (small unilamellar vesicles) to >250 nm (e.g., large multilamellar vesicles). In general, hydrophilic water-soluble drugs are encapsulated in the aqueous core while hydrophobic cargo is incorporated into the lipid bilayer. They may be made from one or multiple phospholipids. In addition to phospholipids, liposomes are often made with additional constituents to enhance stability, increase circulation time, modify drug release as desired, optimize drug loading, achieve specific targeting, and endow them with responsiveness to different pathophysiologic stimuli. These constituents often include, but are not limited to, sterols (e.g., cholesterol), polyethylene glycol (PEG), polymers, and ligands to target specific receptors and stimuli. The nature and ratio of these constituents may be varied and tailored to the properties of the lipids and cargo to be delivered, the desired release profile and the desired degree of targeting/specificity.

Briefly, herein is described a background and method for low pO₂- and pH-dependent drug release from liposomal nanoparticles/microparticles that are sensitive to acidic pH and hypoxia. These liposomal nanoparticles/microparticles can achieve pO₂ sensitivity using a ligand based on, for example, 2-nitroimidazole.

### BRIEF SUMMARY OF THE INVENTION

In one aspect, a nanoparticle is provided. The nanoparticle includes:
(i) at least one of the following:
a hypoxia sensitive ligand of formula I, or an enantiomer, tautomer, or pharmaceutically acceptable salt thereof,

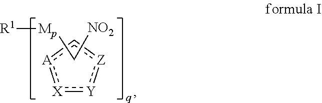

wherein:
   [symbol] represents a single or double bond;
   each occurrence of A, X, Y, and Z is independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ is C₁₋₅₀ alkyl, C₁₋₅₀ alkenyl, or C₁₋₅₀ alkynyl, C₁₋₅₀ alkyl acetamide, C₁₋₅₀ alkenyl acetamide, or C₁₋₅₀ alkynyl acetamide each optionally substituted by at least one substituent selected from the group consisting of OH, OR, N(R)₂, CₙF₂ₙ₋₁, and deuterium (D);
   each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is independently at each occurrence an integer from 0 to 30;
   q is an integer from 1 to 5;
   n is independently at each occurrence an integer from 1 to 10; or
a hypoxia sensitive ligand of formula II, or an enantiomer, tautomer, or pharmaceutically acceptable salt thereof,

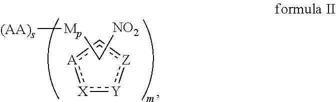

wherein:
   AA is independently at each occurrence a natural or unnatural amino acid, wherein at least one AA is optionally glycosylated by at least one pentose, hexose, or a combination thereof,
   each

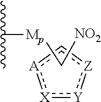

is independently attached to an open valence in (AA)ₛ; (AA)ₛ is linear, branched, or cyclic;
[symbol] represents a single or double bond;
each occurrence of A, X, Y, and Z is independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
Mₚ is optionally substituted by at least one substituent selected from the group consisting of OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is independently at each occurrence an integer from 0 to 30;
s is an integer from 1 to 500;
m is an integer from 1 to 10;
n is independently at each occurrence an integer from 1 to 10; or
a hypoxia sensitive ligand of formula III, or an enantiomer, tautomer, or pharmaceutically acceptable salt thereof,

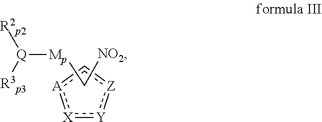

wherein:
   Q is N, CH, or P(═O);
   [symbol] represents a single or double bond;
   A, X, Y, and Z are each independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   each occurrence of R² and R³ is independently selected from the group consisting of H, —O—, —OR, —S—, —S(═O)—, —S(═O)₂—, —SR, —N(R)—, —NR₂, —CR═, —C≡, —CH₂—, —CHR—, —CR₂—, —CH₃, —CH₂—CH₂—, —CH═CH—, —C≡C—, —C(═O)—, and —C(═NR)—;
   R²ₚ₂, R³ₚ₃, and Mₚ are optionally substituted by at least one substituent selected from the group consisting of OH, OR, N(R)₂, CₙF₂ₙ₋₁, and deuterium (D);
   each occurrence of zz is an integer from 2 to 50;
   each R is independently at each occurrence H, F, Cl, Br, I, OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is an integer from 1 to 30;
   p2 is an integer from 1 to 30;
   p3 is an integer from 1 to 30;
   n is independently at each occurrence an integer from 1 to 10; or
a hypoxia sensitive ligand of formula IV, or an enantiomer, tautomer, or pharmaceutically acceptable salt thereof,

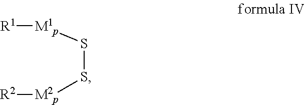

wherein:
   M¹ and M² are each independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ and R² are each pH sensitive lipids;
   each R is independently at each occurrence OH, CF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CF₂ₙ₋₁, and D;
   p is independently at each occurrence an integer from 0 to 30;
   n is independently at each occurrence an integer from 1 to 10; or
a hypoxia sensitive ligand of formula V, or an enantiomer, tautomer, or pharmaceutically acceptable salt thereof, comprising a generation 1, 2, 3, 4, 5, 6, 7, 8, 9, or 10 dendrimer covalently linked to at least one moiety having the structure

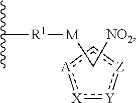

wherein:
   [symbol] represents a single or double bond;
   A, X, Y, and Z are each independently CH, N, NH, O, or S, provided that at least one of A, X, Y, or Z is N, NH, O, or S;
   M is absent, —CH₂—, —O—, —S(═O)—, —SO₂—, —C(═O)—, —C(═O)O—, —OC(═O)—, —C(═O)N(R)—, or —N(R)C(═O)—;
   R¹ is independently at each occurrence C₆₋₅₀ alkyl, C₆₋₅₀ alkenyl, C₆₋₅₀ alkynyl, C₆₋₅₀ alkyl acetamide, C₆₋₅₀ alkenyl acetamide, or C₆₋₅₀ alkynyl acetamide, each optionally substituted by at least one substituent selected from the group consisting of F, Cl, Br, I, OH, OR, N(R)₂, CₙF₂ₙ₋₁, and deuterium (D);
   each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

In one aspect, a method of treating, ameliorating, and/or preventing an ischemic or hypoxic condition in a subject is provided. The method includes administering to the subject in need thereof a therapeutically effective amount of the nanoparticle containing at least one compound of formula I, formula II, formula III, formula IV, or formula V; at least one lipid and at least one hydrophilic therapeutic agent; an outer region and an inner core, and wherein the outer region comprises the at least one lipid and the hypoxia sensitive ligand and the inner core comprises the hydrophilic therapeutic agent.

### BRIEF DESCRIPTION OF THE FIGURES

The drawings illustrate generally, by way of example, but not by way of limitation, various embodiments of the present application.

FIG. 1 is a cartoon of a dual pH and pO₂-sensitive liposome, according to various embodiments. Under healthy physiologic conditions (pH 7.4 and 140 mmHg pO₂, the liposome is intact. Under ischemic conditions, the liposome becomes more permeable, and the rate of cargo release is accelerated. In FIG. 1, C1 (blue diamond) is a hydrophilic therapeutic agent, C2 (black circle) is a hydrophobic agent, C3 is a secondary hydrophilic agent (red circle). These agents may additionally be sensitive to hypoxia, pH, or other stimuli. L1 is a hypoxia sensitive moiety (purple oval) of formula I. L2 is a lipid as described herein. These lipids are optionally conjugated to ligands/agents sensitive to hypoxia, pH, or other stimuli.

FIG. 2 shows NMR analysis of starting materials 2-nitroimidazole and 1-bromooctadecane and the hypoxia sensing product stearyl-2-nitroimidazole (S-2NI), a compound of formula I.

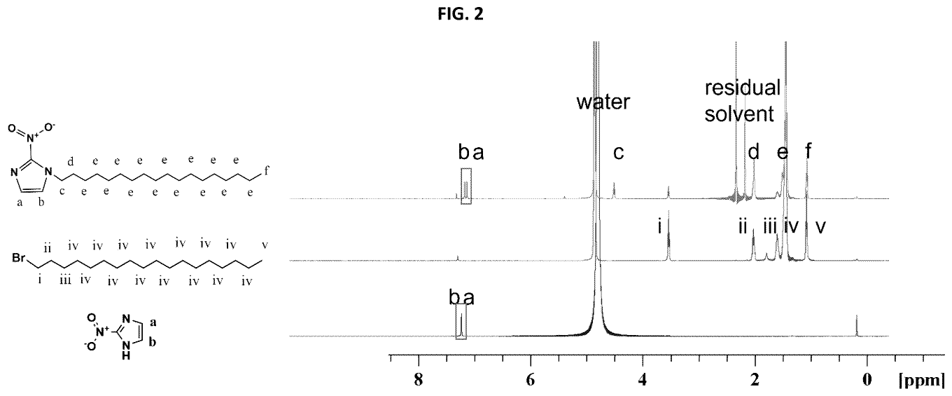

FIGS. 3A-3F show hydrodynamic size distributions of conventional pH sensitive DSPC liposomes and dual targeted pH/hypoxia sensitive liposome nanoparticles (NPs) for conventional saline NPs (FIG. 3A), targeted saline NPs (FIG. 3B), targeted diagnostic NPs (FIG. 3C), targeted metformin NPs (FIG. 3D), targeted pioglitazone NPs (FIG. 3E), and targeted theranostic NPs (FIG. 3F).

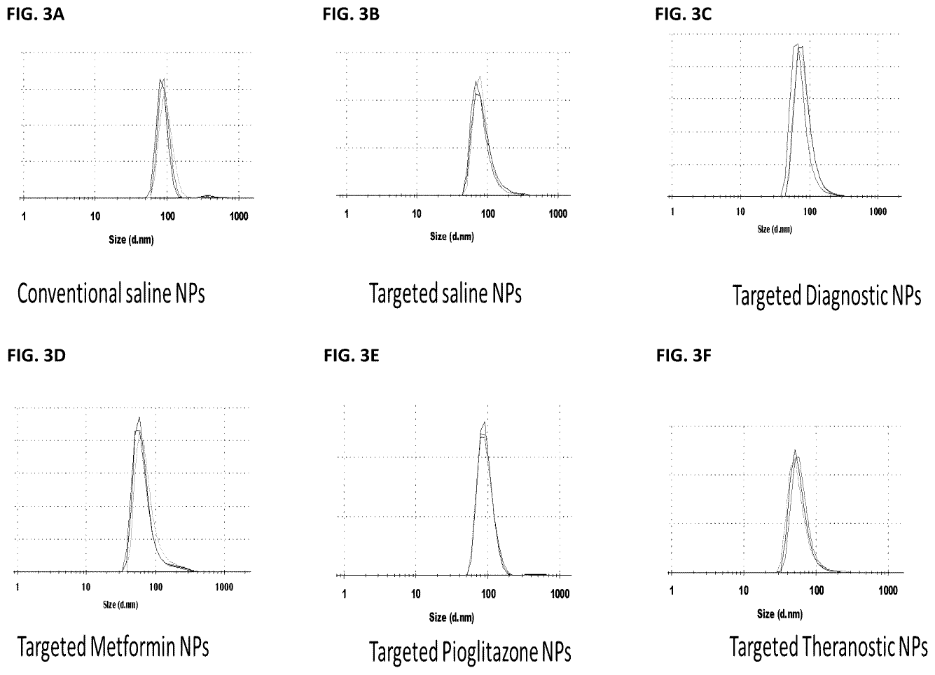

FIGS. 4A-4B show dye release from double-loaded liposomes in saline: FIG. 4A, release rate of FITC-dextran (hydrophilic) from the liposome core at pH 6.85 (orange bars) and pH 7.4 (gray bars) over 7 days. FIG. 4B, release rate of coumarin-6 dye (hydrophobic) from the lipid bilayer at pH 6.85 (orange bars) and pH 7.4 (gray bars) over 7 days. The data shows sustained release over 7 days with relatively higher rates of dye release at the lower pH for both the hydrophilic and hydrophobic model cargo.

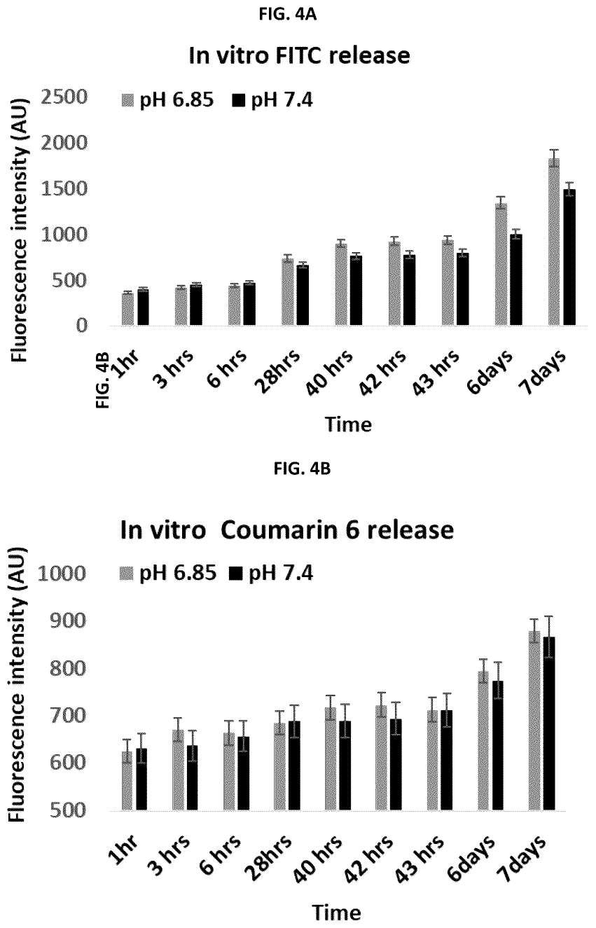

FIG. 5 shows stability of dual targeted pH/hypoxia sensitive liposomes as measured by particle size change over time. The 16-day stability of saline liposomes (checkered bars), therapeutic liposomes containing pioglitazone (grey bars) and theranostic liposomes containing metformin, pioglitazone and gadavist (black bars). The data shows that the sizes for all three liposome types were statistically similar over the 16 days. All DLS measurements were obtained in triplicates on the Malvern ZS90 Zetasizer at 25° C.

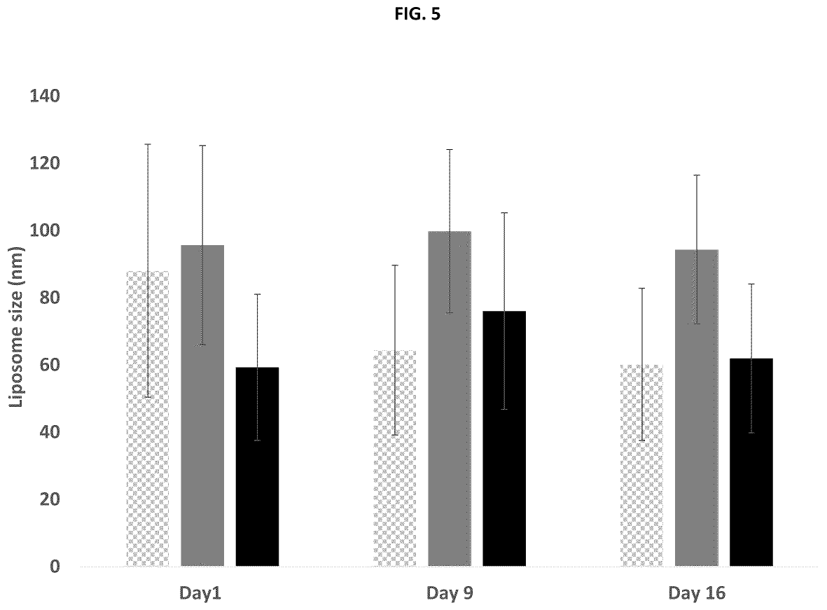

FIGS. 6A-6B show MRI properties of theranostic liposomes at 11.7 T. (FIG. 6A) shows the longitudinal relaxation times (T₁) while (FIG. 6B) shows the transverse relation times (T₂) of phantoms containing various concentrations of gadolinium-based contrast agent (gadavist) in free solution and when encapsulated in theranostic liposomes containing pioglitazone and coumarin-6 at the physiologically relevant pH 7.2. Due to the strong relaxation properties of the nanoparticles, the nanoparticle samples were diluted to 0.1×, 0.25× and 0.5× of the original suspension.

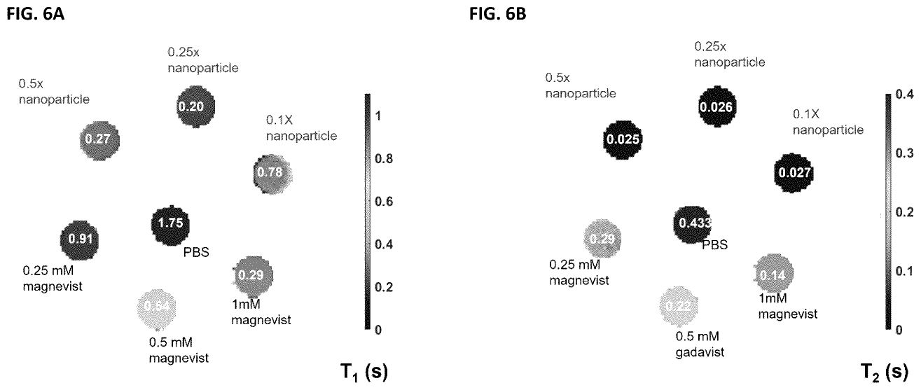

FIG. 7 shows a distribution of dual hypoxia/pH sensitive diagnostic liposomes in MCAO mouse model of ischemic stroke as visualized under T₁-weighted MRI at 11.7T. The liposomes contained the gadolinium-based MRI contrast agent magnevist and were slowly infused through the tail vein (10 L/min).

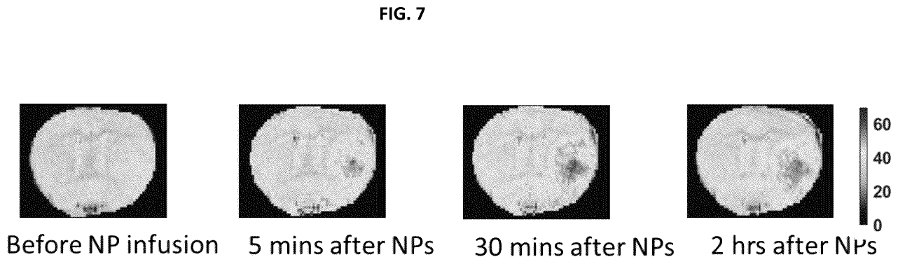

FIG. 8 shows a distribution of dual hypoxia/pH sensitive theranostic liposomes in MCAO mouse model of ischemic stroke as visualized under T₁-weighted MRI at 11.7T. The liposomes contain the gadolinium-based contrast agent gadavist, the fluorescent dye coumarin 6 and the PPARγ agonist drug pioglitazone. The nanoparticles (250 μL) were administered by a series of short bolus IV infusions spread over 5 minutes.

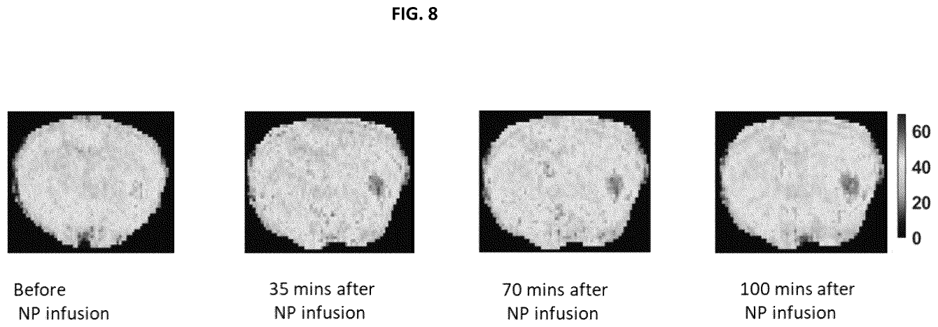

FIGS. 9A-9C show nanocarrier (liposome) formulations by ethanol injection. FIG. 9A shows formulation of liposomes containing hydrophobic cargo (black circles) e.g., pioglitazone, vitamin E and coumarin 6 such that the cargo is incorporated into the lipid bilayer. FIG. 9B shows formulation of liposomes encapsulating hydrophilic cargo (blue diamonds) e.g., metformin, magnevist, FITC-dextran in the liposome core. FIG. 9C shows how double loaded liposomes were formulated to encapsulate both hydrophilic (in the liposome core) and hydrophobic cargo (in the lipid bilayer). All diagnostic and theranostic liposomes contained the dyes FITC-dextran, coumarin-6 and the gadolinium MRI agent magnevist/gadavist. A 2:1 aqueous:organic flow rate ratio (FRR) was used for all liposomes whereby the organic phase was ethanol.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 990.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The substitution can be direct substitution, whereby the hydrogen atom is replaced by a functional group or substituent, or an indirect substitution, whereby an intervening linker group replaces the hydrogen atom, and the substituent or functional group is bonded to the intervening linker group. A non-limiting example of direct substitution is: RR-H→RR-Cl, wherein RR is an organic moiety/fragment/molecule. A non-limiting example of indirect substitution is: RR-H→RR-(LL)_{zz}-Cl, wherein RR is an organic moiety/fragment/molecule, LL is an intervening linker group, and ‘zz’ is an integer from 0 to 100 inclusive. When zz is 0, LL is absent, and direct substitution results. The intervening linker group LL is at each occurrence independently selected from the group consisting of —H, —O—, —OR, —S—, —S(═O)—, —S(═O)₂—, —SR, —N(R)—, —NR₂, —CR═, —C≡, —CH₂—, —CHR—, —CR₂—, —CH₃, —C(═O)—, —C(═NR)—, and combinations thereof (LL)_{zz} can be linear, branched, cyclic, acyclic, and combinations thereof.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂≡C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “heterocycloalkyl” as used herein refers to a cycloalkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C═O group include, but are not limited to, β-propiolactam, γ-butyrolactam, S-valerolactam, and F-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like.

Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxvcyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include

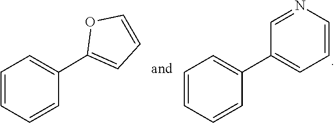 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

As used herein the term “pH sensitive” refers to a molecule which changes in conformation or other properties in response to changes in pH of the surrounding environment.

As used herein, the term further refers to a molecule whose conformation or properties changes as pH decreases from 7.4 to from about pH 6.5 to about pH 3.5.

As used herein, the term “theranostic” refers to an agent that can have or has both diagnostic and therapeutic functions or properties in the same agent/substance/composition. Theranostic agents can be used to simultaneously or sequentially diagnose and treat or ameliorate a particular disease or disorder.

### Preparation of Nanoparticles

Compounds of formula I-formula V or otherwise described herein can be prepared by the general schemes described herein, using the synthetic method known by those skilled in the art. The following examples illustrate non-limiting embodiments of the compound(s) described herein and their preparation. The present disclosure relates to the design, synthesis, formulation, and application of a dual pO₂/pH sensitive liposome-based nanocarriers.

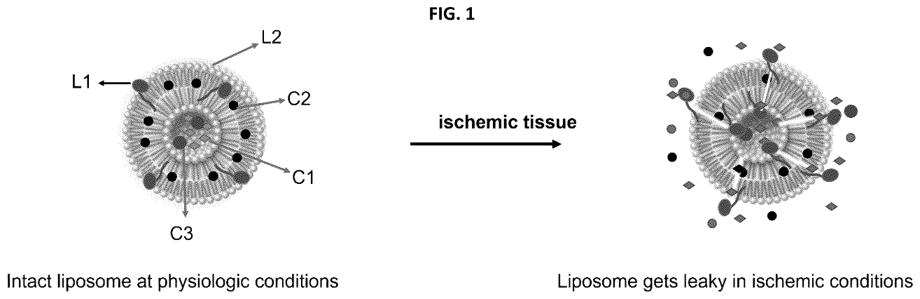

In various embodiments, the nanoparticles described herein are liposomes. As used herein, the terms “nanoparticle” and “nanoparticles” are used interchangeably with the terms “nanocarrier” and “nanocarriers,” respectively. The liposomes can be formulated to form a lipid bi-layer, wherein the hydrophilic lipid heads in one layer face the external environment of the liposome and the hydrophilic lipid heads in the second (inner) layer face the interior. The interior-facing hydrophilic lipid heads and form a hydrophilic core as shown in FIG. 1. The hydrophilic core is suitable to contain one or more hydrophilic therapeutic agents such as drugs and/or theranostic agents, which are released from the liposome upon exposure to a hypoxic environment as described herein. The hydrophobic tails from each of the layers in the bi-layer form an interior hydrophobic region in the liposome that is suitable for containing hydrophobic therapeutic agents. The one or more hydrophobic therapeutic agents are released (within hours to days) from the liposome upon exposure to a hypoxic environment as described herein.

A. Nanoparticles with Hypoxia Sensitive Ligands of Formula I

In various embodiments, a nanoparticle of formula I is provided. In various embodiments, the nanoparticle of formula I includes:
i) a hypoxia sensitive ligand of formula I, or an enantiomer, tautomer, or pharmaceutically acceptable salt thereof,

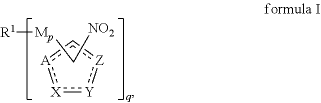

wherein:
   [symbol] represents a single or double bond;
   each occurrence of A, X, Y, and Z is independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ is C₁₋₅₀ alkyl, C₁₋₅₀ alkenyl, or C₁₋₅₀ alkynyl, C₁₋₅₀ alkyl acetamide, C₁₋₅₀ alkenyl acetamide, or C₁₋₅₀ alkynyl acetamide each optionally substituted by at least one substituent selected from the group consisting of OH, OR, N(R)₂, CₙF₂ₙ₋₁, and deuterium (D);
   each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is independently at each occurrence an integer from 0 to 30;
   q is an integer from 1 to 5;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

The nitro (NO₂) group and the -Mₚ-R¹ can be attached at any suitable open valence on the five-membered heterocyclic ring as shown, including C—H and N—H bonds (the open valences). In various embodiments, the NO₂ and Mₚ-R¹ are in a 1,2 relationship on the five-membered heterocyclic ring as shown. In various embodiments, the NO₂ and Mₚ-R¹ are in a 1,3 relationship on the five-membered heterocyclic ring as shown. In various embodiments, R¹ is a C₆₋₅₀ unsaturated group, wherein the unsaturation is at least one carbon-carbon double bond, at least one carbon-carbon triple bond, or a combination thereof. In various embodiments, Mₚ is absent (p=0). In various embodiments q is 1, 2, 3, 4, or 5. In various embodiments, q is 1. In various embodiments, q is 2. In various embodiments, p is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In various embodiments, n is 1, 2, 3, 4, 5, 6, 7, 8, 9, or 10.

In various embodiments, the hypoxia sensitive ligand of formula I is selected from the group consisting of:

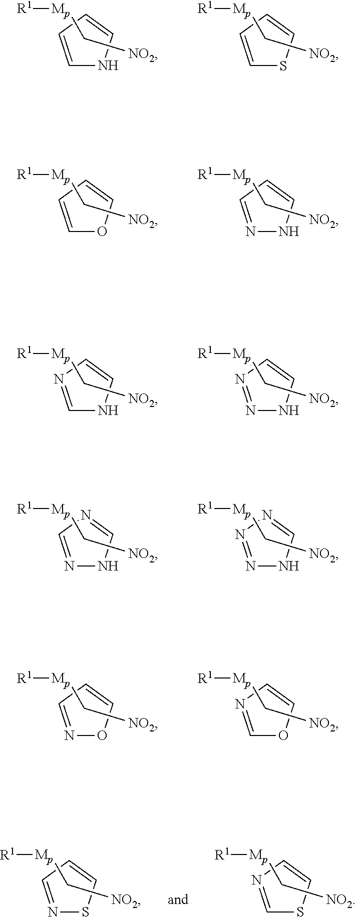

In various embodiments, the hypoxia sensitive ligand of formula I has the structure.

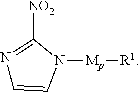

In various embodiments, in the hypoxia sensitive ligand of formula I, R¹ is C₁₂₋₅₀ alkyl optionally substituted by at least one substituent selected from the group consisting of OH, OR, N(R)₂, CₙF₂ₙ₋₁, and deuterium (D). In various embodiments, in the hypoxia sensitive ligand of formula I, R¹ is selected from the group consisting of

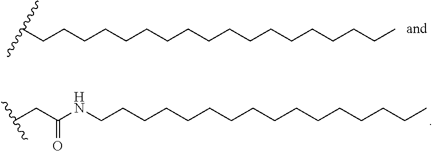 [SMILES: *CCCCCCCCCCCCCCCCCC ; *CC(=O)NCCCCCCCCCCCCCCCC]

In various embodiments, the hypoxia sensitive ligand of formula I has the structure

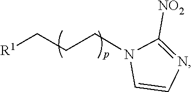

wherein p is an integer from 6 to 24, and R¹ is C₅₀₋₂ₚ alkyl optionally substituted by at least one substituent selected from the group consisting of OH, OR, N(R)₂, CₙF₂ₙ₋₁, and deuterium (D).

In various embodiments, the hypoxia sensitive ligand of formula I is

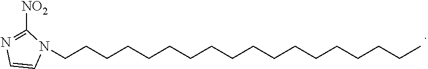 [SMILES: CCCCCCCCCCCCCCCCCCn1ccnc1[N+](=O)[O-]]

In various embodiments, each R or R¹ is independently an alkyl, alkenyl, or alkynyl group containing 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 carbon atoms, optionally substituted by at least one substituent selected from the group consisting of OH, OR, N(R)₂, CₙF₂ₙ₋₁, and deuterium (D).

In some embodiments, the hypoxia sensitive ligands are made from alkyl halide groups of varying chain lengths (C₁₃₋₅₀) and degrees of halogenation (e.g., mono-, di-halogenated, or tri-halogenated). These R and R¹ chains can be linear or branched, saturated or unsaturated (mono or poly unsaturated) whereby the halogenation may be at either ends or any other position (C1-C50) of the linear or branched aryl/aliphatic/alkyl acetamide chain.

Synthesis of the Hypoxia-Sensitive Ligand

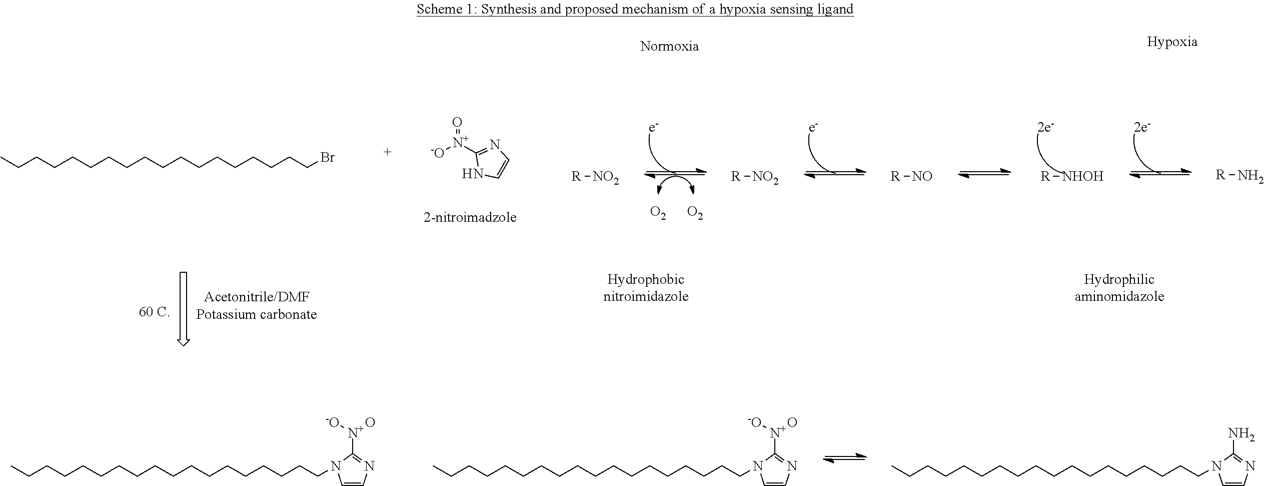

Stearyl-2-nitroimidazole (S-2NI) was synthesized as shown in Scheme 1 by reacting 2-nitroimidazole with equimolar amounts of 1-bromooctadecane and excess potassium carbonate in acetonitrile at 70° C. for 12 hours. The product was dried over vacuum and the resulting solid was then dissolved in dichloromethane/ethyl acetate and washed five times with deionized water. The organic layer was separated in a separatory funnel and dried with sodium sulfate. The final product (S-2NI) was dried in vacuo and stored at −20° C. The same product was also synthesized in DMF instead of acetonitrile with comparable purity and yield.

In various embodiments, when q is greater than 1, multiple

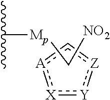

can be covalently attached to a single R¹ chain. Such compounds can be obtained from, for example, reaction with polyhalogenated R¹ groups. For example, in one embodiment, alkylation of erythro-9,10-dibromopentacosane provides a hypoxia sensing ligand of formula I with the structure:

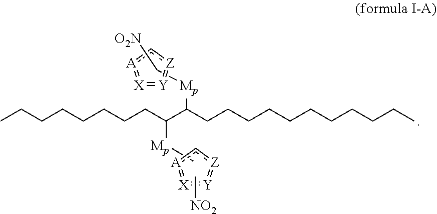

In one embodiment, the hypoxia sensing ligand of formula I has the structure:

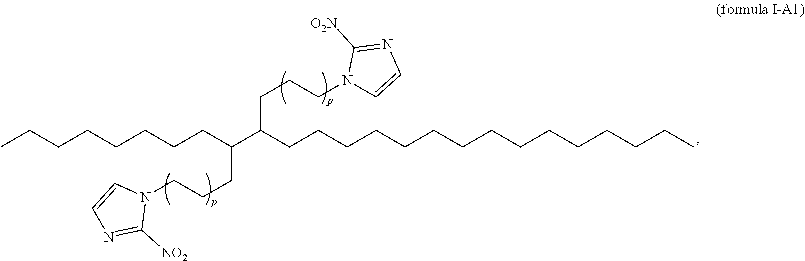

wherein p is independently at each occurrence an integer from 1 to 24.

Analogous derivatives can be obtained, for example according to Scheme 1, with other polyhalogenated R¹ groups. For example, and without limitation, with 1,12-dibromododecane, 18:0 (9,10-dibromo) phosphatidylcholine (PC), 16:0-18:0 (4,5-dibromo) PC, 16:0-18:0 (6,7-dibromo) PC, 16:0-18:0 (9,10-dibromo) PC, or 16:0-18:0 (11,12-dibromo) PC, and the like.

Without being bound by theory, under conditions of low oxygen tension (hypoxia), for example less than about 50, 45, 40, 35, or 30 mmHg of pO₂, the hydrophobic nitro headgroup is reduced, through a reversible electron transfer process, to a hydrophilic amine (as shown in Scheme 1). Since the hydrophilic molecules are repelled from the fatty lipid bilayer of liposomes, the (now hydrophilic) amine headgroup reorients to the aqueous liposome core (inner core) or to the outer surface of the liposome. This change makes the nanoparticle more permeable and the therapeutic agents from both the vesicle core and bilayer can be released at a faster rate selectively within the immediate environment. The reduced aminoimidazole can also covalently bind to macromolecules and proteins in the hypoxic tissue. Consequently, any of the nanoparticles described herein (formulas I-V) can be utilized for pO₂-dependent targeting and drug release.

Without being bound by theory, when the pO₂ (oxygen level) rises (e.g., toward normoxic tissue conditions), the aminoimidazole headgroup converts back to hydrophobic nitroimidazole which then reinserts into the lipid bilayer making the nanoparticle less permeable to releasing therapeutic agents contained in the nanoparticle.

B. Nanoparticles with Hypoxia Sensitive Ligands of Formula II

In various embodiments, a nanoparticle of formula II is provided. In various embodiments, the nanoparticle of formula II includes:
i) a hypoxia sensitive ligand of formula II, or an enantiomer, tautomer, or pharmaceutically acceptable salt thereof,

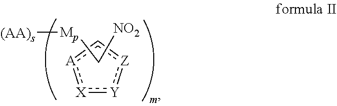

wherein:
   AA is independently at each occurrence a natural or unnatural amino acid, wherein at least one AA is optionally glycosylated by at least one pentose, hexose, or a combination thereof,
   each

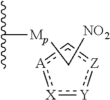

is independently attached to an open valence in (AA)ₛ; (AA)ₛ is linear, branched, or cyclic;
[symbol] represents a single or double bond;
each occurrence of A, X, Y, and Z is independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
Mₚ is optionally substituted by at least one substituent selected from the group consisting of OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of OH, CₙF₂ₙ₋₁, and D;
p is independently at each occurrence an integer from 0 to 30;
s is an integer from 1 to 500;
m is an integer from 1 to 10;
n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

In various embodiments, p is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In various embodiments, n is 1, 2, 3, 4, 5, 6, 7, 8, 9, or 10.

The nitro (NO₂) group and the -Mₚ- can be attached at any suitable open valence on the five-membered heterocyclic ring as shown, including C—H and N—H bonds (the open valences). Any of the five-membered heterocyclic rings described herein with respect to the ligand of formula I can be used with the ligand of formula II. In various embodiments, the NO₂ and Mₚ are in a 1,2 relationship on the five-membered heterocyclic rings as described herein. In various embodiments, the NO₂ and Mₚ are in a 1,3 relationship on the five-membered heterocyclic rings as described herein. In various embodiments, Mₚ is absent (p=0). In various embodiments, p is 1, 2, 3, 4, 5, 6, 7, 8, 9, or 10.

In various embodiments, AA is a natural amino acid. In various embodiments, AA is selected from the group consisting of alanine, arginine, asparagine, aspartic, cysteine, glutamine, glutamic acid, glycine, histidine, isoleucine, leucine, lysine, methionine, phenylalanine, proline, serine, threonine, tryptophan, tyrosine, and valine. In various embodiments, AA is an unnatural amino acid. The type of unnatural amino acid that can be used is not particularly limited, and includes, for example, a D-amino acid, homo amino acid, N-methyl amino acid, alpha-methyl amino acid, beta² amino acid, beta³ amino acid, beta³ homo amino acid, peptoids, aminocyclohexanecarboxylate-derived amino acids, and the like.

In various embodiments, p is 1, and AA is selected from the group consisting of vancomycin, daptomycin, polymix B, volcosporin, pasireotide, dalbavancin, ziconotide, oritavancin, setmelanotide, vasopressin, terlipressin, oxytocin, and cyclosporin.

One or more amino acids (natural or unnatural), can be glycosylated with a pentose or hexose, and the glycosylation can be N-glycosylation, O-glycosylation, or C-glycosylation for any given sugar. Suitable pentoses include, for example, D- or L-isomers of any of arabinose, lyxose, ribose, xylose, xylulose, deoxyribose, and the like. Suitable hexoses include, but are not limited to, D- or L-isomers of any of allose, altrose, glucose, fructose, mannose, gulose, idose, galactose, talose, and the like. Each AA can independently be mono- or poly-glycosylated.

Attachment of each

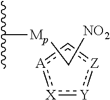

at an open valence in (AA)ₛ can be at any open valence, such as at a CH, NH, OH, or SH moiety.

Ligands of formula II can be made, for example, according to Scheme 2:

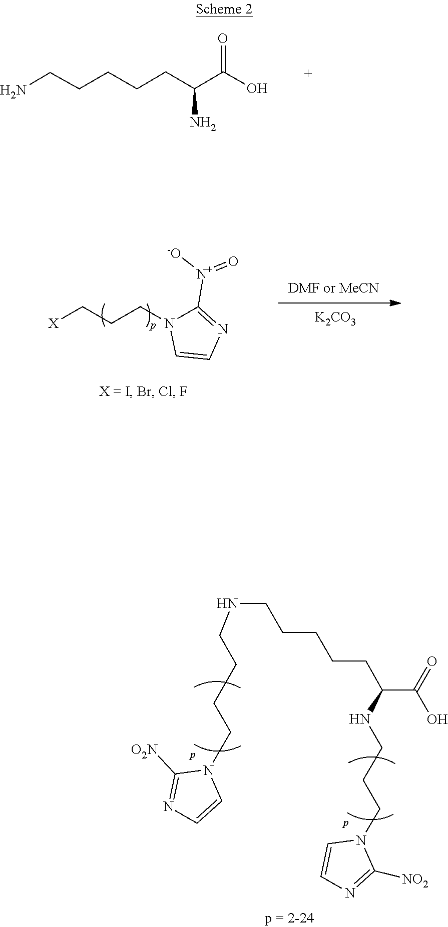

In various embodiments, the hypoxia sensitive ligand of formula II has the structure

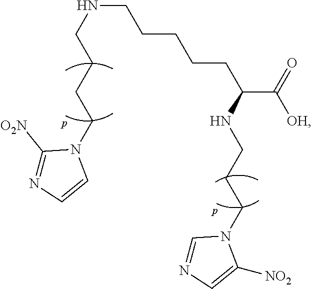

wherein each occurrence of p is independently an integer from 2 to 24.

C. Nanoparticles with Hypoxia Sensitive Ligands of Formula III

In various embodiments, a nanoparticle of formula III is provided. In various embodiments, the nanoparticle of formula III includes:
i) a hypoxia sensitive ligand of formula III, or an enantiomer, tautomer, or pharmaceutically acceptable salt thereof,

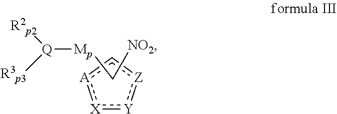

wherein:
   Q is N, CH, or P(═O);
   [symbol] represents a single or double bond;
   A, X, Y, and Z are each independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   each occurrence of R² and R³ is independently selected from the group consisting of H, —O—, —OR, —S—, —S(═O)—, —S(═O)₂—, —SR, —N(R)—, —NR₂, —CR═, —C≡, —CH₂—, —CHR—, —CR₂—, —CH₃, —CH₂—CH₂—, —CH═CH—, —C≡C—, —C(═O)—, and —C(═NR)—;
   R²ₚ₂, R³ₚ₃, and Mₚ are optionally substituted by at least one substituent selected from the group consisting of OH, OR, N(R)₂, CₙF₂ₙ₋₁, and deuterium (D);
   each occurrence of zz is an integer from 2 to 50;
   each R is independently at each occurrence H, F, Cl, Br, I, OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is an integer from 1 to 30;
   p2 is an integer from 1 to 30;
   p3 is an integer from 1 to 30;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

In various embodiments, n is 1, 2, 3, 4, 5, 6, 7, 8, 9, or 10.

In various embodiments, the hypoxia sensitive ligand of formula III has the structure

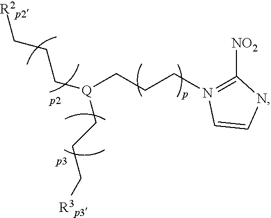

wherein each occurrence of p is an integer from 1 to 24, p2 is an integer from 1 to 24, p3 is an integer from 1 to 24, and p2′ and p3′ are each independently 0 or positive integers less than p2 and p3, respectively. In various embodiments, R²_{p2′} and R³_{p3′} are each optionally substituted by at least one group selected from the group consisting of OH, OR, N(R)₂, CₙF₂ₙ₋₁, and D.

The nitro (NO₂) group and the -Mₚ- can be attached at any suitable open valence on the five-membered heterocyclic ring as shown, including C—H and N—H bonds (the open valences). Any of the five-membered heterocyclic rings described herein with respect to the ligand of formula I can be used with the ligand of formula III. In various embodiments, the NO₂ and Mₚ are in a 1,2 relationship on the five-membered heterocyclic rings as described herein. In various embodiments, the NO₂ and Mₚ are in a 1,3 relationship on the five-membered heterocyclic rings as described herein. In various embodiments, Mₚ is absent (p=0). In various embodiments, each of p, p2, and p3, is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In various embodiments, p2′ and p3′ are independently 0, 1,2, 3, 4, 5, 6, 7, 8, 9, 10, 11, 12, 13, 14, 15, 16, 17, 18, 19, 20, 21, 22, 23, 24, 25, 26, 27, 28, or 29.

In various embodiments, R²ₚ₂ and R³ₚ₃ can be the same or different and represent a targeted/stimuli sensitive ligand made from linear, branched, saturated, unsaturated carbon chain alkyl chain, amine, fatty acid, or peptide. The ligand may be sensitive to hypoxia (based on 2-nitroimidazole or any of the other hypoxia-sensitive moieties described elsewhere in this document) or to other stimuli e.g., pH, receptors (e.g., folate/glutathione/ferritin). The ligand may be targeted to certain transmembrane transporters, e.g., for lactate and glucose.

D. Nanoparticles with Hypoxia Sensitive Ligands of Formula IV

In various embodiments, a nanoparticle of formula IV is provided. In various embodiments, the nanoparticle of formula IV includes:
i) a hypoxia sensitive ligand of formula IV, or an enantiomer, tautomer, or pharmaceutically acceptable salt thereof,

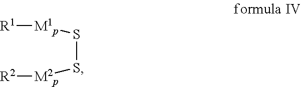

wherein:
   M¹ and M² are each independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ and R² are each pH sensitive lipids, which can contain amino head groups that have potential to become protonated and be positively charged in response to drop in pH;
   each R is independently at each occurrence OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CF₂ₙ₋₁, and D;
   p is independently at each occurrence an integer from 0 to 30;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

In various embodiments, p is independently at each occurrence 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In various embodiments, n is 1, 2, 3, 4, 5, 6, 7, 8, 9, or 10.

The disulfide bond in the ligand of formula IV is known to be sensitive to reductive environments e.g., hypoxia/glutathione whereby it cleaves into two thiols (SH) and increases the permeability of the nanoparticle. In various embodiments, R² and R³ lipids are also sensitive to other stimuli such as pH, and the resulting nanoparticle can release the least one hydrophilic therapeutic agent drug in acidotic and reductive environments present in ischemic strokes and solid tumors.

E. Nanoparticles with Hypoxia Sensitive Ligands of Formula V

In various embodiments, a nanoparticle of formula V is provided. In various embodiments, the nanoparticle of formula V includes:
i) a hypoxia sensitive ligand of formula V, or an enantiomer, tautomer, or pharmaceutically acceptable salt thereof, comprising a generation 1, 2, 3, 4, 5, 6, 7, 8, 9, or 10 dendrimer covalently linked to at least one moiety having the structure

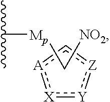

wherein:
   [symbol] represents a single or double bond;
   A, X, Y, and Z are each independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   Mₚ is optionally substituted by at least one substituent selected from the group consisting of OH, OR, N(R)₂, CₙF₂ₙ₋₁, and deuterium (D);
   each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of OH, CₙF₂ₙ₋₁, and D;
   p is independently at each occurrence an integer from 0 to 30;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

In various embodiments, p is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In various embodiments, n is 1, 2, 3, 4, 5, 6, 7, 8, 9, or 10.

In various embodiments, the dendrimer has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 moieties having the structure

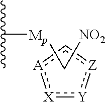

covalently linked to the dendrimer. The covalent linkage can be through any open valence on the dendrimer, such as through one or more NH or OH groups on the dendrimer.

The nitro (NO₂) group and the -Mₚ- can be attached at any suitable open valence on the five-membered heterocyclic ring as shown, including C—H and N—H bonds (the open valences). Any of the five-membered heterocyclic rings described herein with respect to the ligand of formula I can be used with the ligand of formula V. In various embodiments, the NO₂ and Mₚ are in a 1,2 relationship on the five-membered heterocyclic rings as described herein. In various embodiments, the NO₂ and Mₚ are in a 1,3 relationship on the five-membered heterocyclic rings as described herein. In various embodiments, Mₚ is absent (p=0). In various embodiments, p is 1,2, 3, 4, 5, 6, 7, 8, 9, 10, 11, 12, 13, 14, 15, 16, 17, 18, 19, 20, 21, 22, 23, 24, 25, 26, 27, 28, 29, or 30.

In various embodiments, the hypoxia sensitive ligand of formula V, the dendrimer is selected from the group consisting of a polyamideamine (PAMAM) dendrimer, a polypropylamine (POPAM) dendrimer, and a PAMAM-POPAM dendrimer, where beyond 4^{th} generation is unlikely due to solubility limitations.

In various embodiments, the hypoxia sensitive ligand of formula V has the structure

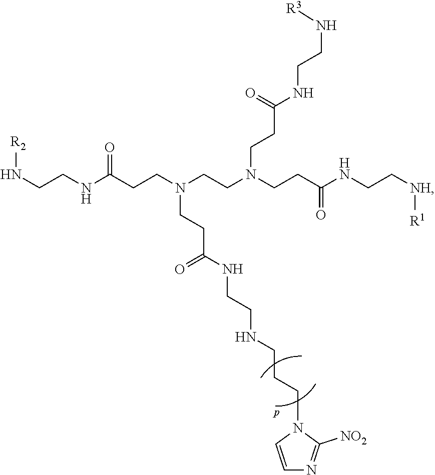

wherein p is an integer from 1 to 20, and wherein R¹, R², and R³ are each independently C₁₋₂₅ alkyl optionally substituted by at least one substituent selected from the group consisting of OH, OR, N(R)₂, CₙF₂ₙ₋₁, and D.

In various embodiments, the hypoxia sensitive ligand of formula I, formula II, formula III, formula IV, or formula V, is present in the nanoparticle in an amount of about 1 to about 20 mol %. In various embodiments, the hypoxia sensitive ligand of formula I, formula II, formula III, formula IV, or formula V, is present in the nanoparticle in an amount of about 1, 2, 3, 4, 5, 6, 7, 8, 9, 10, 11, 12, 13, 14, 15, 16, 17, 18, 19, or about 20 mol %. In various embodiments, any of the liposome-based nanocarriers described herein has a diameter from about 10 to about 1000 nm. In various embodiments, any of the liposome-based nanocarriers described herein has a diameter from about 10, 20, 30, 40, 50, 60, 70, 80, 90, 100, 200, 300, 400, 500, 600, 700, 800, 900, or about 1000 nm. The nanoparticle can be a small unilamellar vesicle or large unilamellar vesicle (LUV), or multilamellar vesicle (MLV). The carrier may also be a microparticle with diameter ranging from about 1 to about 50 microns, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 microns. These larger microparticles can act as drug depots and can be used to deliver therapeutic agents to non-brain tissue.

### Lipid Components in the Nanoparticle

In various embodiments, the nanoparticle containing any one of hypoxia sensitive ligands of formula I, formula II, formula III, formula IV, or formula V, can contain at least one lipid, or an enantiomer or pharmaceutically acceptable salt thereof, is at least one selected from the group consisting of cholesterol, hydrogenated soy L-α-phosphatidylcholine (HSPC), 1,2-dipalmitoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 2-Oleoyl-1-palmitoyl-sn-glycero-3-phosphocholine (POPC), 1,2-distearoyl-sn-glycero-3-phosphocholine (DSPC), 1,2-dilauroyl-sn-glycero-3-phosphorylcholine (DLPC), 1-myristoyl-2-stearoyl-sn-glycero-3-phosphocholine (MSPC), poly(2-methacryloyloxyethyl phosphorylcholine) (PMPC), 1-O-stearoyl-2-O-oleoyl-sn-glycero-3-phosphocholine (SOPC), 1,2-dimyristoyl-sn-glycero-3-phospho-rac-(1-glycerol) (DMPG), 1,2-dilauroyl-sn-glycero-3-phospho-(1′-rac-glycerol) (DLPG), 1,3-dipalmitoyl-sn-glycero-3-phosphoethanolamine (DPPE), 1,2-dimyristoyl-sn-glycero-3-phospho-ethanolamine (DMPE), 1,2-dioleoyl-sn-glycero-3-phosphoethanolamine (DOPE), 1,2-distearoyl-sn-glycero-3-phosphoethanolamine (DSPE), 1,2-distearoyl-sn-glycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-Dimyristoyl-sn-glycero-3-phosphate (DMPA), 1,2-Dipalmitoyl-sn-glycero-3-phosphate (DPPA), 1,2-Distearoyl-sn-glycero-3-phosphate (DSPA), and PEGylated derivatives thereof comprising from 2 to 100 PEG (polyethylene glycol) units. The pH-sensitive lipids will contain amino head groups that have potential to become protonated.

In various embodiments, the at least one lipid can be present in an amount of about 0.5 to about 95 mol %, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 mol %, which can be the total for a single lipid or for multiple lipids. In various embodiments, the at least one lipid is PEGylated with a total of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ethylene glycol units. In various embodiments, at least 1, 2, 3, 4, 5, 6, 7, 8, 9, or 10 different lipids can be present in any of the nanocarriers described herein.

In various embodiments, the at least one lipid is a pH sensitive lipid.

### Hydrophilic Therapeutic Agent

The hydrophilic agent (e.g., C1 in FIG. 1) is not particularly limited in chemical structure of function. In various embodiments, the hydrophilic therapeutic agent is present in an amount of about 0.025 to about 95 mol %, or about 0.025, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 mol %. The amount of the hydrophilic agent can be the amount of a single agent, or the total amount of multiple hydrophilic agents. In various embodiments, the at least one hydrophilic therapeutic agent is at least one selected from the group consisting of metformin, cerebroprotectants, immunosuppressants, immunomodulators, PPAR-γ agonists, antioxidants (e.g. uric acid), alkylating agents, chemotherapeutic agents, anti-inflammatory agents, anti-apoptotic agents (e.g., anti-DAPK1), sulfonylureas (e.g., glipizide, glimepiride, chlorpropamide, glibornuride, gliclazide, glipizide, gliquidone, glisoxepide and glyclopyramide), cerebral vasodilators, neuroprotective peptides, angiogenic growth factors, neurogenic growth factors, oligonucleotides (e.g., antisense oligonucleotide), nucleic acids (e.g., DNA, RNA, siRNA, mRNA), agonists of glycolysis, modulators/antagonists of glycolysis (e.g., 3-bromopyruvate), lactate transporter antagonists, alkaloids, antibiotics, tyrosine kinase inhibitors, and combinations thereof, and the like.

### Hydrophobic Therapeutic Agent

The hydrophobic agent (e.g., C2 in FIG. 1) is not particularly limited in chemical structure of function. In various embodiments, the hydrophobic therapeutic agent is present in an amount of about 0.025 to about 95 mol %, or about 0.025, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 mol %. The amount of the hydrophobic agent can be the amount of a single agent, or the total amount of multiple hydrophobic agents.

In various embodiments, the hydrophobic therapeutic agent is at least one selected from the group consisting of pioglitazone, hydrophobic fluorescent dyes, lipids, sterols, chemotherapeutic agents (e.g. paclitaxel), and combinations thereof. In various embodiments, the hydrophobic fluorescent dye is FITC (fluorescein isothiocyanate)-dextran, although any other suitable fluorescent dye can be used.

In various embodiments, the nanocarrier comprises at least two hydrophilic therapeutic agents or at least one hydrophilic therapeutic agent and at least one hydrophobic therapeutic agent chosen from PPARγ agonists, PPARα agonists, steroidal anti-inflammatory drug, non-steroidal anti-inflammatory drug, thiazolidinediones, sulfonylureas, statins, biguanides, antiapoptotic agents, antioxidants, rho-associated protein kinases, and poly-ADP ribose polymerase (PARP) inhibitors.

In some embodiments, the at least one hydrophilic therapeutic agent is at least one selected from the group consisting of pioglitazone, metformin, uric acid, fasudil, glyburide, glipizide, fingolimod, Vitamin E, veliparib, olaparib, rucaparib, 3-aminobenzamide, pamiparib, talazoparib, lovastatin, simvastatin, and combinations thereof.

In various embodiments, one or more of the hydrophilic agents are encapsulated in a cyclodextrin prior to being encapsulated in the nanocarriers described herein. Suitable cyclodextrins include, but are not limited to, α-cyclodextrin (α-CD), β-cyclodextrin (β-CD), γ-cyclodextrin (γ-CD), Hydroxyethyl-β-CD (HE-β-CD), Hydroxypropyl-β-CD (HP-β-CD), Sulfobutylether-β-CD (SBE-β-CD), Methyl-β-CD (M-β-CD), Dimethyl-β-CD (DM-β-CD), (DIMEB), Randomly dimethylated-β-CD (RDM-β-CD), Randomly methylated-β-CD (RM-β-CD), Carboxymethyl-β-CD (CM-β-CD), Carboxymethyl ethyl-β-CD (CME-β-CD), Diethyl-β-CD (DE-β-CD), Tri-O-methyl-β-(CD TRIMEB), Tri-O-ethyl-β-CD (TE-β-CD), Tri-O-butyryl-β-CD (TB-β-CD), Tri-O-valeryl-β-CD (TV-β-CD), Di-O-hexanoyl-β-CD (DH-β-CD), Glucosyl-β-CD (G1-β-CD), Maltosyl-β-CD (G2-β-CD), and 2-hydroxy-3-trimethyl-ammoniopropyl-β-CD (HTMAPC).

### Contrast Agents

In various embodiments, any of the nanocarriers described herein can contain at least one contrast agent. In various embodiments, the contrast agent is at least one selected from the group consisting of a transition metal-containing contrast agent, iron oxide-containing contrast agent, iodinated CT agents, PET radioisotopes, radioactive agents, fluorophores, quantum dots, and chemiluminescent agents.

The contrast agent can aid in the detection, staging, diagnosis and monitoring of lesions, inflammation, edema, membrane integrity or tumors and can include clinically approved and non-approved contrast enhancers for magnetic resonance imaging (MRI) or their derivatives, for example:

I. Gd-based agents such as, for example, gadavists, magnevist, dotarem, gadobenate, gadobutrol, gadoterate, gadoteridol, gadodiamide, eovist, gadofosveset, gadoxetic acid, ablavar and the like. The agents include linear or macrocyclic chelates and can have varying charges (−5 to +5). The agents include single monomers, dimers, trimers, or dendrimers. The agents can also include biologically relevant lanthanide and transition metal complexes including, but not limited to, those of Tm, Eu, Dy, Yb, Zn, Co, Cr, Fe, Cu, Mn, Ni, Ga. The generated MRI contrast agent may can be based on traditional longitudinal and transverse relaxation enhancements (T1, T2) or chemical saturation transfer (CEST) or chemical shift spectroscopic imaging (MRS/CSI) or biosensor imaging of redundant deviation (BTRDS) in shifts using paramagnetic or diamagnetic agents.

II. The contrast agent can include iron oxides, including iron oxide microparticles, superparamagnetic iron oxides (SPIOs), ferrites, spinel ferrites, e.g., Molday ION, ferrumoxytol.

III. The contrast agents can also include non-MRI e.g., iodinated CT agents, PET radioisotopes, radiotracers/agents, fluorophores, quantum dots and chemiluminescent agents.

In various embodiments, one or more of the contrast agents cab be covalently linked to the hypoxia sensitive ligand of formula I, formula II, formula III, formula IV, or formula V, or to at least one lipid as described herein.

### Second Hydrophilic Agent

In various embodiments, the inner core further comprises a second hydrophilic therapeutic agent. In various embodiments, the second hydrophilic therapeutic agent enhances the permeability of the blood-brain barrier (BBB) to the nanocarrier. For example, the second hydrophilic therapeutic agent is an A2A adenosine receptor agonist such as regadenoson (Lexiscan®). In various embodiments, the nanocarriers described herein can also include a brain efflux suppressing agent, such as a β-glycoprotein inhibitor (e.g., erythromycin, ritonavir).

### Nanocarrier Formulations

In various embodiments, a therapeutic nanocarrier composition includes:
about 30-60 mol % DSPC or HSPC (helper lipids);
about 10-30 mol % cholesterol;
about 5-20 mol % pH sensitive lipid;
about 1-20 mol % hypoxia sensitive lipid moiety;
about 0.5-15% PEGylated lipid;
about 0.5-15% pioglitazone,
about 0.01-1 mol % fluorescent dye,
about 0.5-5 mol % magnevist/gadavist.

In some embodiments, HSPC, DPPC, DOPC, DMPC, POPC, and other phosphocholine phospholipids described herein, or mixtures thereof, were used in place of DSPC as the main helper lipids depending on desired stability/release profile.

In various embodiments, the nanocarriers include metformin as they hydrophilic therapeutic agents, or both pioglitazone (hydrophobic therapeutic agent) and metformin, each independently in an amount of about 0.5 to about 20 mol % as the main therapeutic cargo.

In various, the formulation is adjusted to achieve desired drug loading, stability, targeting, stimuli responsiveness and cargo release. Such formulations may be selected to include the following and mixtures thereof at desired ratios:
0.025 mol % to 90 mol % drug/theranostic agent
0.5 to 95 mol % helper lipid or mixture of helper lipids (e.g., DSPC, HSPC, DOPC, DPPC, DDPC, DMPC, DLPC, MMPC, MSPC, PMPC, SOPC, DMPG, DLPG, DPPE, DMPE, DOPE, DSPE, DSPS, DPPS, DMPS, DMPA, DPPA and DSPA
0.1 mol % to about 50 mol % cholesterol, PEGylated cholesterol/other sterols/PEGylated sterols
0.1 mol % to about 50 mol % medium chain and long chain mono/di/triglycerides,
0.1 to 50 mol % pH sensitive lipid/polymer,
0.1 to 50 mol % hypoxia sensitive lipid/polymer,
0.1 to 50 mol % ionizable lipid
0.1 to 50 mol % hypoxia lipid/polymer lipids conjugated with MRI/NMR/PET/CT/US contrast/tracer/sensitizing agents and dyes,
0.1 to 50 mol % lipid/polymer lipids conjugated to drugs, targeting ligands and antibodies,
0.1 to 50 mol % PEGylated lipid/polymer (containing polyethylene glycol, PEG) and pharmaceutically acceptable excipients. The liposomes may be composed of some or all these constituents.
0.1 to 50 mol % pH responsive and disulfide cleavable lipids.

Non limiting examples of disulfide cleavable lipids include Coatsome SS-EC, Coatsome SS-OP, Coatsome SS-E, Coatsome SS-OC, and the like. The nanocarriers may be composed of some or all these constituents. In some embodiments, these lipids contain pH-responsive tertiary amines and disulfide bonds that cleave in reductive environments.

Referring to FIG. 1, in some embodiments, C1 is a primary hydrophilic agent that is selected from a preventative/protective/therapeutic drug/prodrug, C2 is a hydrophobic agent, drug, dye, contrast agent, sensitizing agent or combinations thereof, C3 is a secondary hydrophilic agent that may be selected from a second drug, dye, contrast agent, radiotracer, sensitizing agent or combinations thereof. These agents may additionally be sensitive to hypoxia, pH or other stimuli. Referring to FIG. 1, in some embodiments, L1 is a hypoxia sensitive moiety based on 2-nitroimidazole but can also be selected from nitroazoles, nitrothiazoles, nitrooxazoles, nitrofurans, and other nitroimidazoles; L2 can be a phospholipid, pegylated lipid, cholesterol, vitamin E, sterols, fatty acid, medium-long chain mono/di/triglyceride or mixtures thereof that is optionally conjugated to ligands/agents sensitive to hypoxia, pH, or other stimuli.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

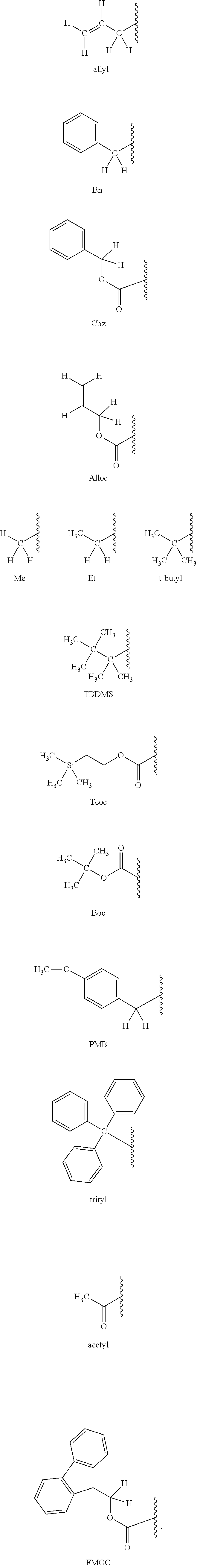

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treating Hypoxic or Ischemic Conditions

The nanocarriers described herein, including pO₂ and pO₂/pH liposome-based nanocarriers, are effective for use in the detection and/or treatment of pathologies/conditions characterized by ischemia, which features hypoxic and acidic environments.

The disclosure includes a method of treating a disease or disorder caused by a hypoxic or ischemic condition using the nanocarriers of formula I, formula II, formula III, formula IV, or formula V, and pharmaceutical compositions thereof. Non-limiting examples of a hypoxic or ischemic condition include systemic ischemia, ischemic stroke, transient ischemic stroke, traumatic brain injury, organ ischemia, chemically-induced ischemia, spinal cord injury, brain contusion, concussion, and solid tumor.

In various embodiments, the solid tumor is at least one tissue or organ selected from the group consisting of brain, head, neck, liver, spleen, kidney, lung, skin, pancreas, breast, cervical, testicular, ovarian, eye, oral, rectum, bladder, prostate, stomach, and colon.

In various embodiments, the administration is a bolus infusion or a continuous infusion.

Cerebral ischemia (tissue acidosis and hypoxia) is a hallmark of ischemic stroke and other pathologies such as traumatic brain injury and solid tumors. The nanocarriers described herein can be used to deliver high payloads specifically into stroke infarcts. The nanocarriers (liposomes) described herein displayed sustained pH-sensitive drug release (higher release rates at lower pH), high physical stability, were well tolerated in vivo and preferentially accumulated in the infarcted brain tissue(hypoxic) compared to the healthy brain in mouse models of ischemic stroke. These liposome nanoparticles were used for detection, non-invasive diagnosis, targeting, tracking, and monitoring of drug delivery and therapy efficacy in ischemic stroke. Beyond strokes, the nanocarriers described herein can be used in a broad range of pathologies characterized by ischemia including traumatic brain injury, solid tumors, organ ischemia, brain contusions and main others as described elsewhere in this invention.

In some embodiments the nanocarriers described herein are useful for the treatment of stroke, TBI, concussions, spinal cord injury, brain contusions, shock, or tissue/systemic trauma/ischemia.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats a disease or disorder caused by a hypoxic or ischemic condition.

In certain embodiments, administering the compound(s) described herein to the subject allows for administering a lower dose of the additional therapeutic agent as compared to the dose of the additional therapeutic agent alone that is required to achieve similar results in treating a a disease or disorder caused by a hypoxic or ischemic condition in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating a disease or disorder caused by a hypoxic or ischemic condition. These additional therapeutic agents may comprise compounds that are commercially available or synthetically accessible to those skilled in the art. These additional therapeutic agents are known to treat or reduce the symptoms, of a a disease or disorder caused by a hypoxic or ischemic condition.

In certain embodiments, the compounds described herein can be used in combination with radiation therapy. In other embodiments, the combination of administration of the compounds described herein and application of radiation therapy is more effective in treating or preventing a disease or disorder caused by a hypoxic or ischemic condition than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder caused by a hypoxic or ischemic condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a disease or disorder caused by a hypoxic or ischemic condition in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder caused by a hypoxic or ischemic condition in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder caused by a hypoxic or ischemic condition in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a disease or disorder caused by a hypoxic or ischemic condition in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### Examples

Various embodiments of the present application can be better understood by reference to the following Examples which are offered by way of illustration. The scope of the present application is not limited to the Examples given herein.

### Materials and Methods

### Materials

The lipids and cholesterol used in this study were purchased from Avanti Polar Lipids, Inc. (Alabaster, AL). Cholesterol and the lipids used on this study—1,2-distearoyl-sn-glycero-3-phosphocholine (DSPC), dipalmitoyl phosphatidylcholine (DPPC), 2-dioleoyl-sn-glycero-3-phosphocholine (DOPC), L-α-phosphatidylcholine, hydrogenated soy PC (HSPC), 1,2-dipalmitoyl-sn-glycero-3-succinate (16:0 DGS), 1,2-dimyristoyl-sn-glycero-3-phosphoethanolamine-N-[methoxy(polyethylene glycol)-2000] (14:0 PEG2000 PE), N-palmitoyl homocysteine (PHC). 1-bromooctadecane, 2-nitroimidazole, potassium carbonate, acetonitrile, metformin, pioglitazone, vitamin E, coumarin 6, FITC-dextran of varying MWs (3-70 KDa), sucrose and HPLC-grade ethanol were purchased from SigmaAldrich. was purchased from Fisher Scientific (Waltham, MA). The hollow fiber tangential flow filtration (TFF) membranes (MicroKros 20 cm², 500 KDa and 50 nm, MPES, 0.5 mm), Regenerated cellulose (RC) dialysis tubing (3500 Da MWCO), Float-A-Lyzer G2 dialysis device, was purchased from Repligen Corporation (Waltham, MA). All other chemicals were commercial products of reagent grade and were used without further purification.

### 2.1 Synthesis of Hypoxia Responsive Conjugate

The hypoxia sensitive conjugate (stearyl-2-nitroimidazole) was synthesized by conjugating 2-nitroimidazole to 1-bromooctadecane. 1 g, (3 mmol) of 1-bromooctadecane was mixed with slight excess (0.37 g, 3.3 mmol) of 2-nitroimidazole (1.1×) in acetonitrile and excess (4×) potassium carbonate (1.66 g) and reacted for 24 hours at 60° C. The product was extracted over ethyl acetate then washed and separated three times with DI water in a separatory funnel. The organic layer was dried with sodium sulfate then filtered and the residual solvent dried on a rotary evaporator to obtain the product stearyl-2-nitroimidazole. Product identity was confirmed by NMR and mass spectroscopy.

### Preparation of Nanocarriers

Liposomes were made by ethanol injection, thin film hydration or remote loading as appropriate for the drug. While there are several methods of making liposomes thin film hydration and ethanol injection were used in this study. Drugs were loaded into the liposomes by active or passive loading methods. In passive loading methods, the drug is entrapped during the liposome formation step. In active loading, the drug is inserted into preformed liposomes. While the passive loading methods involve fewer steps, significantly higher drug entrapment can be achieved with active loading methods especially for drugs with low but pH-dependent water solubilities (weak bases and weak acids).

### Ethanol Injection Method

### Saline-containing Nanocarriers

Liposomes were formulated by the ethanol injection method as previously described. Briefly, a lipid mixture of DSPC:cholesterol:PEG-PE (75:20:5 mol %) was dissolved in 2 ml anhydrous ethanol, sonicated and filtered through a 0.22 μm syringe filter. Another syringe containing 10 ml of 150 mM saline was also made. The two syringes were then connected to a T-junction and placed on two opposite syringe pumps and the flow rate set such that the flow rate ratio was (FRR) was 5:1 saline:ethanolic lipid solution (5:1 ml/min). The two pumps were then run simultaneously. A translucent suspension of liposomes formed spontaneously and was filtered by tangential flow filtration (TFF) five times to remove all traces of ethanol using 150 mM filtered saline for buffer exchange. In the final diafiltration cycle, the filtered liposomes were diluted to 5 mL. Different liposome formulations were also made using different lipids (e.g., using DPPC or HSPC in place of DSPC or by including the pH-sensitive lipids (PHC or DGS) and the oxygen-sensitive stearyl-2-nitroimidazole (S-2NI) ligand as desired. In the dual pH/hypoxia sensitive liposomes, a lipid composition of DSPC:Chol:DGS:S-2NI:14:0 PEG-PE (62.5:12.5:10:10:5 mol % in ethanol was mixed with saline as described above.

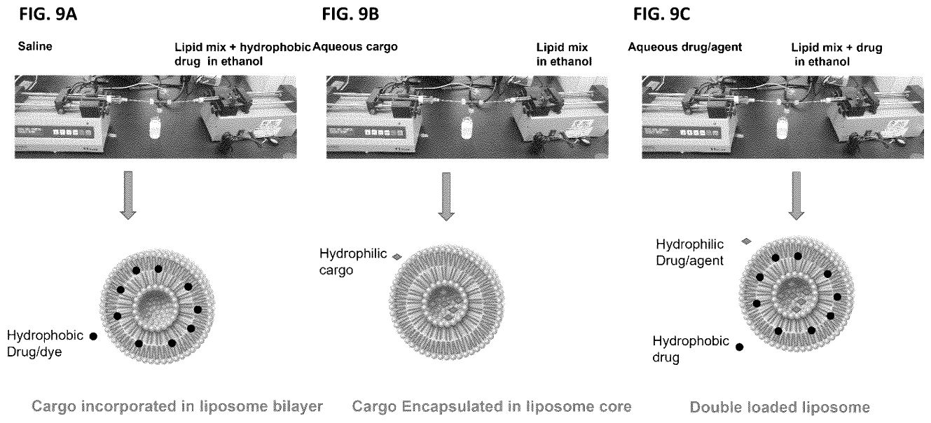

FIGS. 9A-9C show a non-limiting setup for formulating various types of nanocarriers (liposomes) by the ethanol injection method, according to some embodiments. The identity and ratios of the lipids can be varied as desired to optimize stability, targeting, drug loading, and drug release, as described in detail herein in various embodiments.

To incorporate the hydrophobic agents (in the lipid bilayer of the liposomes) e.g., pioglitazone, coumarin-6 dye, or vitamin E, the agents were co-dissolved with the lipids in ethanol and mixed with the aqueous solutions as shown in FIG. 9A.

### Pioglitazone-containing Nanocarriers

8 mg of pioglitazone was mixed with the lipid mixture and dissolved in 2 ml of anhydrous ethanol dissolved (such that the pioglitazone was (4 mg/mL) and mixed with 10 ml of pH 7.2 saline through opposite ends of the T-junction. A flow rate ratio of 5:1 mg/mL saline:ethanolic drug-lipid solution was used. Theranostic pioglitazone liposomes were made similarly but using 0.5 mmol/ml gadavist or magnevist solution instead of saline. The resulting translucent liposome suspension was filtered by TFF (using pH 7.2 saline as the exchange buffer) to remove ethanol and any unencapsulated metformin and MRI contrast agents. Any hydrophobic drug could be used in place or in addition to pioglitazone in this method. The drug:lipid ratio can also be varied to encapsulate desired agent/drug dose.

### Metformin-containing Nanocarriers

200 mM metformin solution in saline was mixed with ethanolic lipids through the T-junction as shown in FIG. 9B at a flow rate ratio of 5:1 metformin:lipidic ethanol solution. To make theranostic liposomes, the MRI contrast agent (magnevist or gadavist (0.5 mmol/ml)), and or the fluorescent dye FITC-dextran the agent was dissolved in saline and mixed with the ethanolic lipid solution through a T-Junction as shown in FIG. 9B. A translucent suspension of liposomes formed spontaneously and was filtered by TFF through five diafiltration cycles in saline to remove any unencapsulated drugs. Any hydrophilic drug/agent could be used in place or in addition to metformin in this method. The drug:lipid ratio can also be varied to encapsulate desired agent/drug dose, in accordance with various embodiments.

### Fluorescent Nanocarriers

The hydrophobic fluorescent dye coumarin-6 was mixed with the lipid mix and dissolved in anhydrous ethanol. Another solution containing the fluorescent dye FITC-dextran (MW=3-50 KDa) in pH 7.2 saline was then made and the two solutions mixed at high pressure through the T-junction. The flow rate ratio of the aqueous:ethanolic solution was 5:1. The resulting yellow-green fluorescent liposomes were then filtered by TFF to remove ethanol and any free un-encapsulated dye. Any fluorescent dye used in place or in addition to FITC-dextran and coumarin-6 in this method. The dye:lipid ratio can also be varied to achieve the desired fluorescence, in accordance with various embodiments.

Fluorescent theranostic liposomes containing the hydrophilic, hydrophobic or both hydrophilic and hydrophobic drugs were made similarly. The hydrophilic drug was co-dissolved with FITC-Dextran in pH 7.2 saline while the hydrophobic drug (e.g., pioglitazone, Vitamin E, etc.) was mixed with the lipids, coumarin 6 and dissolved in anhydrous ethanol. The two solutions were then mixed together at a 5:1 aqueous:organic flow rate ration through a T-junction and the resulting liposomes filtered by TFF to remove any free drugs, dyes, and ethanol. pH 7.2 saline was used as the exchange buffer for TFF.

### Double-Loaded Nanocarriers

For double-loaded liposomes incorporating one or multiple hydrophilic and hydrophobic agents, the hydrophilic agents were dissolved in saline while the hydrophobic agents were dissolved in the ethanolic lipid solution and mixed through the T-junction as shown FIG. 9C. The resulting liposomes were then filtered five times by TFF to remove ethanol and any unencapsulated drugs/agents and the final filtered liposomes diluted to 5 mL.

### Thin Film Hydration Method for Preparing Nanocarriers

Metformin-containing liposomes: A lipid mixture (DSPC:Chol:DGS:S-2NI:14:0 PEG-PE (62.5:12.5:10:10:5 mol %) was dissolved in anhydrous ethanol and dried under vacuum in a rotary evaporator for 24 hours at 35° C. to create a thin lipid film. The film was then hydrated with 200 mg/mL metformin in pH 7.2 phosphate buffered saline and sonicated at 60° C. The composition and ratios of the lipid mix can be varied as desired. Theranostic liposomes were made similarly using an aqueous solution containing metformin (200 mg/mL) and 0.5 mmol/mL gadavist (or magnevist). The resulting suspension was subjected to 11 cycles of freezing and thawing using liquid nitrogen and a 60° C. water bath. The thawed suspension was vortexed before being frozen at each cycle. After 11 cycles, the final suspension was extruded 15 times at 60° C. through polycarbonate filters with 200 nm pores then filtered by TFF to remove any un-encapsulated metformin and contrast agents. A higher amount of metformin could be entrapped by adding a 10 mg/mL metformin solution in methanol into the lipid solution to make a metformin/lipid film then hydrated with a solution of 200 mg/mL metformin in saline.

Pioglitazone-containing liposomes: Pioglitazone (16 mg) was mixed with 100 mg of the lipid mixture and dissolved together in ethanol. The ethanolic lipid solution was then dried under vacuum for 24 hours at 35° C. to create a thin lipid/drug film. The film was then hydrated with saline and sonicated at 60° C. Theranostic liposomes were made similarly with the drug/lipid film using an aqueous solution containing 0.5 mmol/ml gadavist (or magnevist) instead of saline. The resulting suspension was subjected to 11 cycles of freezing and thawing using liquid nitrogen and a 60° C. water bath. The thawed suspension was vortexed before being frozen at each cycle. After 11 cycles, the final suspension was extruded 15 times at 60° C. through polycarbonate filters with 200 nm pores then filtered by TFF to remove any un-encapsulated metformin and contrast agents.

### Active-Loaded Nanocarriers

To investigate the effect of formulation method on liposome properties, metformin and pioglitazone liposomes were made passively by ethanol injection (as described above) and by remote/active loading. For the case of active loading of metformin, calcium acetate liposomes were made by the ethanol injection method by mixing 250 mM calcium acetate in distilled water (pH 4) with the ethanolic lipids solution and using TFF to remove any un-encapsulated calcium acetate. Ammonium acetate can also be used in place of calcium acetate. The resulting filtered liposomes were then incubated in a 50 mL solution of 0.5 mg/mL metformin at 60° C. for 1 hour (metformin concentration can be varied). The liposome-drug mixture was then cooled to room temperature and filtered by TFF again to remove any free unencapsulated metformin. Pioglitazone was loaded remotely in a similar manner but using ammonium sulfate instead of calcium acetate. The resulting ammonium sulfate liposomes were filtered by TFF then incubated in a 0.1 mg/mL acidified solution of pioglitazone (pH 1.2) in 5% ethanol:distilled water (v/v) and stirred at 60° C. for 1 hour. The liposome suspension was then cooled to room temperature and filtered by TFF to remove ethanol and any free pioglitazone.

### Characterization

The chemical structure of the synthesized hypoxia sensitive product stearyl-2-nitroimidazole was determined by NMR on a Bruker 500 MHz vertical bore spectrometer in deuterated chloroform (CDCl₃) and compared against the spectra of the starting materials as shown in FIGS. 9A-9C. The identity of the product was also confirmed by mass spectroscopy on the Agilent 6120 quadrupole LC/MS System.

The size (hydrodynamic diameter) and charge (zeta potential) of the liposome nanoparticles was determined by dynamic light scattering (DLS) and electrophoretic scattering, respectively on the Malvern ZS90 Zetasizer (Malvern Instruments, Malvern UK) after diluting the samples 10× in 150 mM saline. All measurements were carried out in triplicates.

### Statistical Analysis

All measurements were determined in triplicates and the data are expressed as mean standard deviation (SD) or standard error of the mean (SEM). For the different time points and formulation groups (single independent variable), one-way ANOVA would be used to evaluate the differences.

We will make ischemia-responsive liposomes by the ethanol injection method. Briefly, lipid mixtures will be dissolved in ethanol and mixed with saline/5% sucrose (1:5 flow rate ratio) at high pressure through opposite ends of a T-junction. Hydrophobic drugs will be co-dissolved with the ethanolic lipids to incorporate them into the liposome bilayer, while hydrophilic drugs will be co-dissolved with the aqueous solution to encapsulate them into the liposome core. To make double-loaded liposomes, the hydrophobic drugs will be co-dissolved with the lipids in ethanol, the hydrophilic drug dissolved in saline and the two solutions mixed at high pressure through the T junction to make liposomes. The resulting liposomes will then be filtered five times by tangential flow filtration to remove any unencapsulated drugs using 300 kDa mPES microKros filters (Repligen Inc., Waltham MA). The aqueous solution will be made to contain the MRI agent magnevist (theranostic liposomes) to enable tracking of liposomes distribution with MRI. All liposomes will be characterized for size, charge and stability by dynamic light scattering and drug loading by HPLC.

All mice will be anesthetized with ˜1-1.5% isoflurane. Breathing rate will be measured by a respiration pad under the torso, and temperature will be monitored through a rectal fiber-optic thermometer and maintained at 37° C. MRI data will be acquired on a 9.4T horizontal-bore Bruker Avance system using 2 cm surface/4 cm volume coil. Diffusion-weighted imaging (DWI, specifically apparent diffusion coefficient or ADC) can distinguish acute ischemic lesion from ventricular cerebral spinal fluid (CSF). An optimized MRI protocol includes Rapid Imaging with Refocused Echoes (RARE) imaging for anatomy plus a combination of T2 and ADC to differentiate the stroke lesion from CSF in ventricles. Although conventional MRI is not sensitive to acute hemorrhage, there are some prospects with multi-modal MRI. Whether or not lysis occurs in RBCs, deoxyhemoglobin is paramagnetic and results in enough signal loss on T2* weighted sequences or susceptibility weighted imaging (SWI), with a blooming artifact. Combining T2 (edema) and ADC (infarction) with T2* weighted MRI can show if hemorrhage is present.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:

Embodiment 1 provides a nanoparticle comprising at least one of the following:
i) at least one hypoxia sensitive ligand selected from:
a hypoxia sensitive ligand of formula I, or an enantiomer, tautomer, or pharmaceutically acceptable salt thereof,

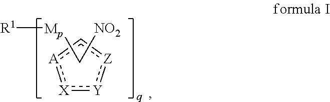

wherein:
   [symbol] represents a single or double bond;
   each occurrence of A, X, Y, and Z is independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ is C₁₋₅₀ alkyl, C₁₋₅₀ alkenyl, or C₁₋₅₀ alkynyl, C₁₋₅₀ alkyl acetamide, C₁₋₅₀ alkenyl acetamide, or C₁₋₅₀ alkynyl acetamide each optionally substituted by at least one substituent selected from the group consisting of OH, OR, N(R)₂, CₙF₂ₙ₋₁, and deuterium (D); each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is independently at each occurrence an integer from 0 to 30;
   q is an integer from 1 to 5;
   n is independently at each occurrence an integer from 1 to 10; or
a hypoxia sensitive ligand of formula II, or an enantiomer, tautomer, or pharmaceutically acceptable salt thereof,

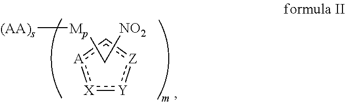

wherein:
   AA is independently at each occurrence a natural or unnatural amino acid, wherein at least one AA is optionally glycosylated by at least one pentose, hexose, or a combination thereof,
   each

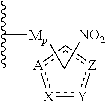

is independently attached to an open valence in (AA)ₛ; (AA)ₛ is linear, branched, or cyclic;
[symbol] represents a single or double bond;
each occurrence of A, X, Y, and Z is independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
Mₚ is optionally substituted by at least one substituent selected from the group consisting of OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is independently at each occurrence an integer from 0 to 30;
s is an integer from 1 to 500;
m is an integer from 1 to 10;
n is independently at each occurrence an integer from 1 to 10; or
a hypoxia sensitive ligand of formula III, or an enantiomer, tautomer, or pharmaceutically acceptable salt thereof,

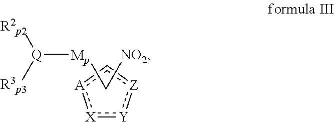

wherein:
   Q is N, CH, or P(═O);
   [symbol] represents a single or double bond;
   A, X, Y, and Z are each independently CH, N, NH, O, or S, provided that at least one of A, X, Y, or Z is N, NH, O, or S;
   each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   each occurrence of R² and R³ is independently selected from the group consisting of H, —O—, —OR, —S—, —S(═O)—, —S(═O)₂—, —SR, —N(R)—, —NR₂, —CR═, —C≡, —CH₂—, —CHR—, —CR₂—, —CH₃, —CH₂—CH₂—, —CH═CH—, —C≡C—, —C(═O)—, and —C(═NR)—;
   R²ₚ₂, R³ₚ₃, and Mₚ are optionally substituted by at least one substituent selected from the group consisting of OH, OR, N(R)₂, CₙF₂ₙ₋₁, and deuterium (D);
   each occurrence of zz is an integer from 2 to 50;
   each R is independently at each occurrence H, F, Cl, Br, I, OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is an integer from 1 to 30;
   p2 is an integer from 1 to 30;
   p3 is an integer from 1 to 30;
   n is independently at each occurrence an integer from 1 to 10; or
a hypoxia sensitive ligand of formula IV, or an enantiomer, tautomer, or pharmaceutically acceptable salt thereof,

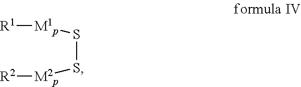

wherein:
   M¹ and M² are each independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
   R¹ and R² are each pH sensitive lipids;
   each R is independently at each occurrence OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   p is independently at each occurrence an integer from 0 to 30;
   n is independently at each occurrence an integer from 1 to 10; or a hypoxia sensitive ligand of formula V, or an enantiomer, tautomer, or pharmaceutically acceptable salt thereof, comprising a generation 1, 2, 3, 4, 5, 6, 7, 8, 9, or 10 dendrimer covalently linked to at least one moiety having the structure

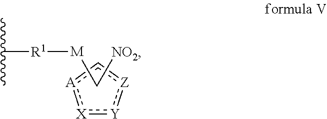

wherein:
   [symbol] represents a single or double bond;
   A, X, Y, and Z are each independently CH, N, NH, O, or S, provided that at least one of A, X, Y, or Z is N, NH, O, or S;
   M is absent, —CH₂—, —O—, —S(═O)—, —SO₂—, —C(═O)—, —C(═O)O—, —OC(═O)—, —C(═O)N(R)—, or —N(R)C(═O)—;
   R¹ is independently at each occurrence C₆₋₅₀ alkyl, C₆₋₅₀ alkenyl, C₆₋₅₀ alkynyl, C₆₋₅₀ alkyl acetamide, C₆₋₅₀ alkenyl acetamide, or C₆₋₅₀ alkynyl acetamide, each optionally substituted by at least one substituent selected from the group consisting of F, Cl, Br, I, OH, OR, N(R)₂, CₙF₂ₙ₋₁, and deuterium (D);
   each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
   n is independently at each occurrence an integer from 1 to 10;
ii) at least one lipid and at least one hydrophilic therapeutic agent;
iii) an outer region and an inner core, and
   wherein the outer region comprises the at least one lipid and the hypoxia sensitive ligand and the inner core comprises the hydrophilic therapeutic agent.

Embodiment 2 provides the nanoparticle of embodiment 1, wherein the hypoxia sensitive ligand of formula I is selected from the group consisting of.

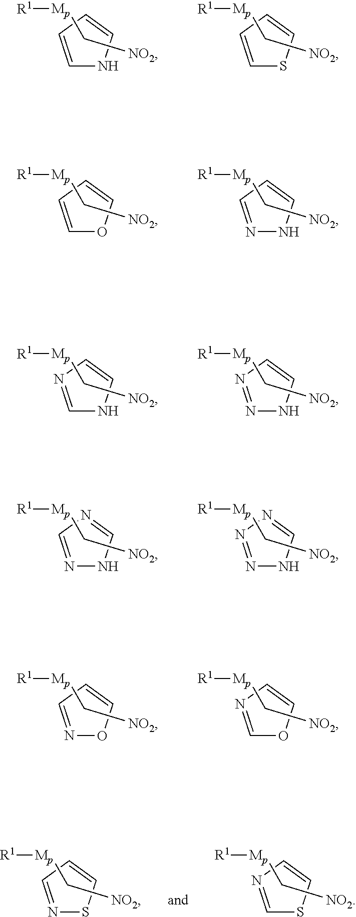

Embodiment 3 provides the nanoparticle of any one of embodiments 1-2, wherein the hypoxia sensitive ligand of formula I is

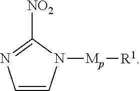

Embodiment 4 provides the nanoparticle of any one of embodiments 1-3, wherein in the hypoxia sensitive ligand of formula I, R¹ is a C₁₂₋₅₀ alkyl optionally substituted by at least one substituent selected from the group consisting of OH, OR, N(R)₂, CₙF₂ₙ₋₁, and deuterium (D).

Embodiment 5 provides the nanoparticle of any one of embodiments 1-4, wherein in the hypoxia sensitive ligand of formula I, formula II, formula III, formula IV, or formula V, M is absent.

Embodiment 6 provides the nanoparticle of any one of embodiments 1-5, wherein in the hypoxia sensitive ligand of formula I, R¹ is selected from the group consisting of

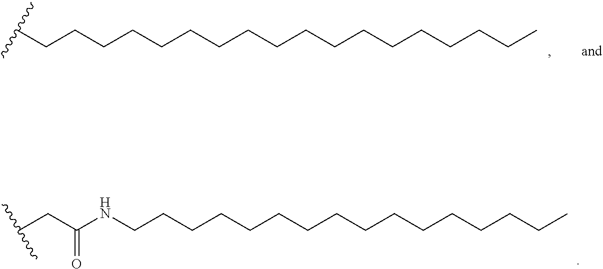 [SMILES: *CCCCCCCCCCCCCCCCCC ; *CC(=O)NCCCCCCCCCCCCCCCC]

Embodiment 7 provides the nanoparticle of any one of embodiments 1-6, wherein the hypoxia sensitive ligand of formula I has the structure

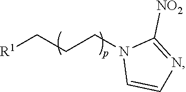

wherein p is an integer from 6 to 24, and R¹ is a C₅₀₋₂ₚ alkyl optionally substituted by at least one substituent selected from the group consisting of OH, OR, N(R)₂, CF₂ₙ₋₁, and deuterium (D).

Embodiment 8 provides the nanoparticle of any one of embodiments 1-7, wherein the hypoxia sensitive ligand of formula I is

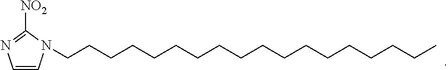 [SMILES: CCCCCCCCCCCCCCCCCCn1ccnc1[N+](=O)[O-]]

Embodiment 9 provides the nanoparticle of any one of embodiments 1,-8 wherein the hypoxia sensitive ligand of formula I, formula II, formula III, formula IV, or formula V, is present in an amount of about 1 to about 20 mol %.

Embodiment 10 provides the nanoparticle of any one of embodiments 1-9, wherein in the hypoxia sensitive ligand of formula II, AA is selected from the group consisting of alanine, arginine, asparagine, aspartic, cysteine, glutamine, glutamic acid, glycine, histidine, isoleucine, leucine, lysine, methionine, phenylalanine, proline, serine, threonine, tryptophan, tyrosine, and valine.

Embodiment 11 provides the nanoparticle of any one of embodiments 1-10, wherein in the hypoxia sensitive ligand of formula II, p is 1 and (AA)ₚ is selected from the group consisting of vancomycin, daptomycin, polymix B, volcosporin, pasireotide, dalbavancin, ziconotide, oritavancin, setmelanotide, vasopressin, terlipressin, oxytocin, and cyclosporin.

Embodiment 12 provides the nanoparticle of any one of embodiments 1-11, wherein the hypoxia sensitive ligand of formula II has the structure

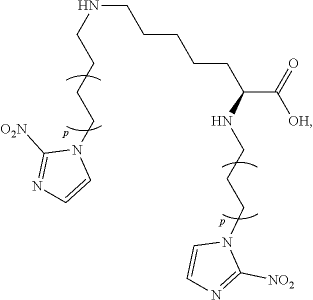

wherein each occurrence of p is independently an integer from 2 to 24.

Embodiment 13 provides the nanoparticle of any one of embodiments 1-12, wherein the hypoxia sensitive ligand of formula III has the structure

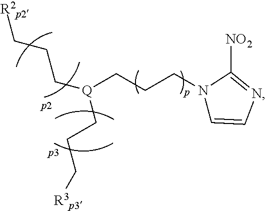

wherein p is an integer from 1 to 24, p2 is an integer from 1 to 24, p3 is an integer from 1 to 24, p2′ is an integer from 0 to 23, p3′ is an integer from 0 to 23, and R²_{p2′} and R³_{p3′} are each independently optionally substituted by at least one group selected from the group consisting of OH, OR, N(R)₂, CₙF₂ₙ₋₁, and D.

Embodiment 14 provides the nanoparticle of any one of embodiments 1-13, wherein in the hypoxia sensitive ligand of formula V, the dendrimer is selected from the group consisting of a polyamideamine (PAMAM) dendrimer, a polypropylamine (POPAM) dendrimer, and a PAMAM-POPAM dendrimer.

Embodiment 15 provides the nanoparticle of any one of embodiments 1-14, wherein the hypoxia sensitive ligand of formula V has the structure

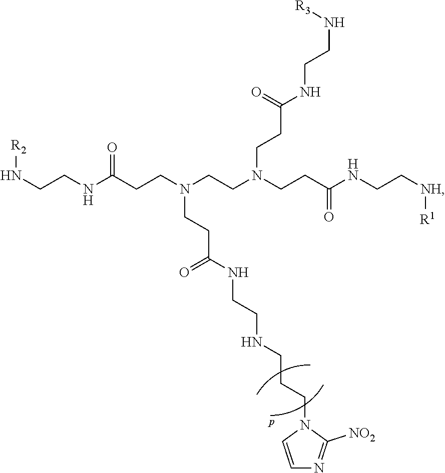

wherein p is an integer from 1 to 20, and wherein R¹, R², and R³ are each independently C₁₋₂₅ alkyl optionally substituted by at least one substituent selected from the group consisting of OH, OR, N(R)₂, CₙF₂ₙ₋₁, and D.

Embodiment 16 provides the nanoparticle of any one of embodiments 1-15, wherein the at least one lipid, or an enantiomer or pharmaceutically acceptable salt thereof, is at least one selected from the group consisting of cholesterol, hydrogenated soy L-α-phosphatidylcholine (HSPC), 1,2-dipalmitoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 2-Oleoyl-1-palmitoyl-sn-glycero-3-phosphocholine (POPC), 1,2-distearoyl-sn-glycero-3-phosphocholine (DSPC), 1,2-dilauroyl-sn-glycero-3-phosphorylcholine (DLPC), poly(2-methacryloyloxyethyl phosphorylcholine) (PMPC), 1-O-stearoyl-2-O-oleoyl-sn-glycero-3-phosphocholine (SOPC), 1,2-dimyristoyl-sn-glycero-3-phospho-rac-(1-glycerol) (DMPG), 1,2-dilauroyl-sn-glycero-3-phospho-(1′-rac-glycerol) (DLPG), 1,3-dipalmitoyl-sn-glycero-3-phosphoethanolamine (DPPE), 1,2-dimyristoyl-sn-glycero-3-phospho-ethanolamine (DMPE), 1,2-dioleoyl-sn-glycero-3-phosphoethanolamine (DOPE), 1,2-distearoyl-sn-glycero-3-phosphoethanolamine (DSPE), 1,2-distearoyl-sn-glycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-Dimyristoyl-sn-glycero-3-phosphate (DMPA), 1,2-Dipalmitoyl-sn-glycero-3-phosphate (DPPA), 1,2-Distearoyl-sn-glycero-3-phosphate (DSPA), and PEGylated derivatives thereof comprising from 2 to 100 PEG units.

Embodiment 17 provides the nanoparticle of any one of embodiments 1-16, wherein the at least one lipid is at least one pH sensitive lipid.

Embodiment 18 provides the nanoparticle of any one of embodiments 1-17, wherein the at least one lipid is independently present in an amount of about 0.5 to about 95 mol %.

Embodiment 19 provides the nanoparticle of any one of embodiments 1-18, wherein the at least one hydrophilic therapeutic agent is present in an amount of about 0.025 to about 95 mol %.

Embodiment 20 provides the nanoparticle of any one of embodiments 1-19, wherein the at least one hydrophilic therapeutic agent is at least one selected from the group consisting of metformin, cerebroprotectants, immunosuppressants, immunomodulators, PPAR-γ agonists, antioxidants, alkylating agents, chemotherapeutic agents, anti-inflammatory agents, anti-apoptotic agents, sulfonylureas, cerebral vasodilators, neuroprotective peptides, angiogenic growth factors, neurogenic growth factors, oligonucleotides, nucleic acids, agonists of glycolysis, modulators/antagonists of glycolysis, lactate transporter antagonists, alkaloids, antibiotics, tyrosine kinase inhibitors, and combinations thereof.

Embodiment 21 nanoparticle any one of embodiments 1-20, wherein the outer region further comprises at least one hydrophobic therapeutic agent.

Embodiment 22 provides the nanoparticle of any one of embodiments 1-21, wherein the at least one hydrophobic therapeutic agent is at least one selected from the group consisting of pioglitazone, hydrophobic fluorescent dyes, lipids, sterols, chemotherapeutic agents, and combinations thereof.

Embodiment 23 provides the nanoparticle of any one of embodiments 1-22, wherein the at hydrophobic therapeutic agent is independently present in an amount of about 0.5 to about 95 mol %.

Embodiment 24 provides the nanoparticle of any one of embodiments 1-23, wherein the nanoparticle comprises at least two hydrophilic therapeutic agents or at least one hydrophilic therapeutic agent and at least one hydrophobic therapeutic agent chosen from PPARγ agonists, PPARα agonists, steroidal anti-inflammatory drug, non-steroidal anti-inflammatory drug, thiazolidinediones, sulfonylureas, statins, biguanides, antiapoptotic agents, antioxidants, rho-associated protein kinases, and poly-ADP ribose polymerase (PARP) inhibitors.

Embodiment 25 provides the nanoparticle of any one of embodiments 1-24, wherein the at least one hydrophilic therapeutic agent is encapsulated in a cyclodextrin.

Embodiment 26 provides the nanoparticle of any one of embodiments 1-25, wherein the at least one hydrophilic therapeutic agent is at least one selected from the group consisting of pioglitazone, metformin, uric acid, fasudil, glyburide, glipizide, fingolimod, Vitamin E, veliparib, olaparib, rucaparib, 3-aminobenzamide, pamiparib, talazoparib, lovastatin, simvastatin, and combinations thereof.

Embodiment 27 provides the nanoparticle of any one of embodiments 1-26, further comprising a contrast agent.

Embodiment 28 provides the nanoparticle of any one of embodiments 1-27, wherein the contrast agent is at least one selected from the group consisting of a transition metal-containing contrast agent, iron oxide-containing contrast agent, iodinated CT agents, PET radioisotopes, radioactive agents, fluorophores, quantum dots, and chemiluminescent agents.

Embodiment 29 provides the nanoparticle of any one of embodiments 1-28, wherein the contrast agent is covalently linked to the hypoxia sensitive ligand of formula I, formula II, formula III, formula IV, or formula V.

Embodiment 30 provides the nanoparticle of any one of embodiments 1-29, wherein the inner core further comprises a second hydrophilic therapeutic agent.

Embodiment 31 provides the nanoparticle of any one of embodiments 1-30, wherein the second hydrophilic therapeutic agent enhances the permeability of the blood-brain barrier (BBB) to the nanoparticle.

Embodiment 32 provides the nanoparticle of any one of embodiments 1-31, wherein the second hydrophilic therapeutic agent is an A2A adenosine receptor agonist.

Embodiment 33 provides the nanoparticle of any one of embodiments 1-32, wherein the second hydrophilic therapeutic agent is regadenoson.

Embodiment 34 provides the nanoparticle of any one of embodiments 1-33, further comprising a brain efflux suppressing agent.

Embodiment 35 provides the nanoparticle of any one of embodiments 1-34, wherein the brain efflux suppressing agent is a P-glycoprotein inhibitor.

Embodiment 36 provides a method of treating an ischemic or hypoxic condition in a subject, the method comprising:
administering to the subject in need thereof a therapeutically effective amount of the nanoparticle of claim 1.

Embodiment 37 provides the method of embodiment 36, wherein the ischemic or hypoxic condition is as a result of a condition selected from the group consisting of systemic ischemia, ischemic stroke, transient ischemic stroke, traumatic brain injury, organ ischemia, chemically-induced ischemia, spinal cord injury, brain contusion, concussion, and solid tumor.

Embodiment 38 provides the method of any one of embodiments 36-37, wherein the solid tumor is at least one tissue or organ selected from the group consisting of brain, head, neck, liver, spleen, kidney, lung, skin, pancreas, breast, cervical, testicular, ovarian, eye, oral, rectum, bladder, prostate, stomach, and colon.

Embodiment 39 provides the method of any one of embodiments 36-38, wherein the administration is by a route of administration selected from the group consisting of intravenous (IV), intraarterial, intraperitoneal, subcutaneous, intradermal, retroorbital, direct injection, convection enhanced delivery, intrathecal, intranasal, inhalers, sublingual, and oral administration.

Embodiment 40 provides the method of any one of embodiments 36-39, wherein the administration is a bolus infusion or a continuous infusion.

Embodiment 41 provides the method of any one of embodiments 36-40, further comprising administering an additional therapeutic agent.

Embodiment 42 provides the method of any one of embodiments 36-41, wherein the additional therapeutic agent is administered concurrently or sequentially with the nanoparticle.

What is claimed is:

1. A nanoparticle comprising at least one of the following:
i) at least one hypoxia sensitive ligand selected from:
a hypoxia sensitive ligand of formula I, or an enantiomer, tautomer, or pharmaceutically acceptable salt thereof, 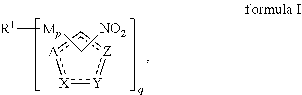
wherein: [symbol]
represents a single or double bond;
each occurrence of A, X, Y, and Z is independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
R¹ is C₁₋₅₀ alkyl, C₁₋₅₀ alkenyl, or C₁₋₅₀ alkynyl, C₁₋₅₀ alkyl acetamide, C₁₋₅₀ alkenyl acetamide, or C₁₋₅₀ alkynyl acetamide each optionally substituted by at least one substituent selected from the group consisting of OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is independently at each occurrence an integer from 0 to 30;
q is an integer from 1 to 5;
n is independently at each occurrence an integer from 1 to 10; or
a hypoxia sensitive ligand of formula II, or an enantiomer, tautomer, or
pharmaceutically acceptable salt thereof, 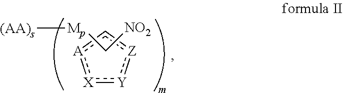
wherein:
AA is independently at each occurrence a natural or unnatural amino acid, wherein at least one AA is optionally glycosylated by at least one pentose, hexose, or a combination thereof;
each 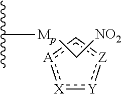
is independently attached to an open valence in (AA)ₛ;
(AA)ₛ is linear, branched, or cyclic; [symbol]
represents a single or double bond;
each occurrence of A, X, Y, and Z is independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
Mₚ is optionally substituted by at least one substituent selected from the group consisting of OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is independently at each occurrence an integer from 0 to 30;
s is an integer from 1 to 500;
m is an integer from 1 to 10;
n is independently at each occurrence an integer from 1 to 10; or
a hypoxia sensitive ligand of formula III, or an enantiomer, tautomer, or pharmaceutically acceptable salt thereof, 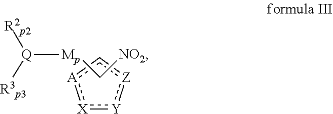
wherein:
Q is N, CH, or P(═O); [symbol]
represents a single or double bond;
A, X, Y, and Z are each independently CH, N, NH, O, or S, provided that at least one of A, X, Y, or Z is N, NH, O, or S;
each occurrence of M is independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
each occurrence of R² and R³ is independently selected from the group consisting of H, —O—, —OR, —S—, —S(═O)—, —S(═O)₂—, —SR, —N(R)—, —NR₂, —CR═, —C≡, —CH₂—, —CHR—, —CR₂—, —CH₃, —CH₂—CH₂—, —CH═CH—, —C≡C—, —C(═O)—, and —C(═NR)—;
R²ₚ₂, R³ₚ₃, and Mₚ are optionally substituted by at least one substituent selected from the group consisting of OH, OR, N(R)₂, CₙF₂ₙ₋₁, and deuterium (D);
each occurrence of zz is an integer from 2 to 50;
each R is independently at each occurrence H, F, Cl, Br, I, OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is an integer from 1 to 30;
p2 is an integer from 1 to 30;
p3 is an integer from 1 to 30;
n is independently at each occurrence an integer from 1 to 10; or
a hypoxia sensitive ligand of formula IV, or an enantiomer, tautomer, or pharmaceutically acceptable salt thereof, 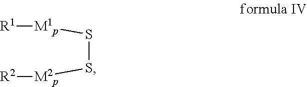
wherein:
M¹ and M² are each independently absent (a bond), —CH₂—, —CH₂—CH₂—, —CH═CH—, —C≡C—, —O—, —S(═O)—, —SO₂—, —C(═O)—, —C(═O)O—, —OC(═O)—, —C(═O)N(R)—, or —N(R)C(═O)—;
R¹ and R² are each pH sensitive lipids;
each R is independently at each occurrence OH, CₙF₂ₙ₋₁, D, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
p is independently at each occurrence an integer from 0 to 30;
n is independently at each occurrence an integer from 1 to 10; or
a hypoxia sensitive ligand of formula V, or an enantiomer, tautomer, or pharmaceutically acceptable salt thereof, comprising a generation 1, 2, 3, 4, 5, 6, 7, 8, 9, or 10 dendrimer covalently linked to at least one moiety having the structure 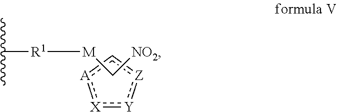
wherein: [symbol]
represents a single or double bond;
A, X, Y, and Z are each independently CH, N, NH, O, or S, provided that at least one of A, X, Y, or Z is N, NH, O, or S;
M is absent, —CH₂—, —O—, —S(═O)—, —SO₂—, —C(═O)—, —C(═O)O—, —OC(═O)—, —C(═O)N(R)—, or —N(R)C(═O)—;
R¹ is independently at each occurrence C₆₋₅₀ alkyl, C₆₋₅₀ alkenyl, C₆₋₅₀ alkynyl, C₆₋₅₀ alkyl acetamide, C₆₋₅₀ alkenyl acetamide, or C₆₋₅₀ alkynyl acetamide, each optionally substituted by at least one substituent selected from the group consisting of F, Cl, Br, I, OH, OR, N(R)₂, CₙF₂ₙ₋₁, and deuterium (D);
each R is independently at each occurrence H, C₁₋₅₀ alkyl, C₂₋₅₀ alkenyl, or C₂₋₅₀ alkynyl, wherein each alkyl, alkenyl, or alkynyl group is optionally substituted by at least one substituent selected from the group consisting of F, Cl, Br, I, OH, CₙF₂ₙ₋₁, and D;
n is independently at each occurrence an integer from 1 to 10;
ii) at least one lipid and at least one hydrophilic therapeutic agent; and
iii) an outer region and an inner core, wherein the outer region comprises the at least one lipid and the hypoxia sensitive ligand and the inner core comprises the hydrophilic therapeutic agent;
optionally wherein the hypoxia sensitive ligand of formula I, formula II, formula III, formula IV, or formula V, is present in an amount of about 1 to about 20 mol % in the nanoparticle.

2. The nanoparticle of claim 1, wherein the hypoxia sensitive ligand of formula I is selected from the group consisting of:

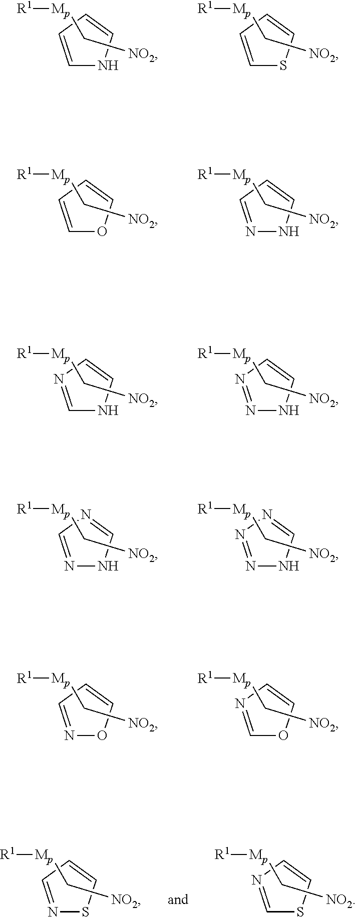

3. The nanoparticle of claim 2, wherein the hypoxia sensitive ligand of formula I is

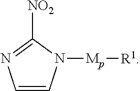

4. The nanoparticle of claim 1, wherein in the hypoxia sensitive ligand of formula I, R¹ is a C₁₂₋₅₀ alkyl optionally substituted by at least one substituent selected from the group consisting of OH, OR, N(R)₂, CₙF₂ₙ₋₁, and deuterium (D).

5. The nanoparticle of claim 1, wherein M is absent in the hypoxia sensitive ligand of formula I, formula II, formula III, formula IV, or formula V.

6. The nanoparticle of claim 1, wherein in the hypoxia sensitive ligand of formula I, R¹ is selected from the group consisting of

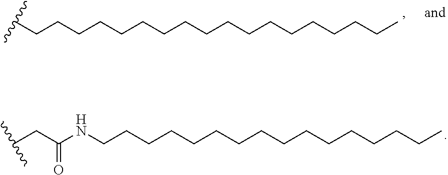 [SMILES: *CCCCCCCCCCCCCCCCCC ; *CC(=O)NCCCCCCCCCCCCCCCC]

7. The nanoparticle of claim 1, wherein the hypoxia sensitive ligand of formula I has the structure 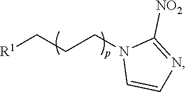
wherein p is an integer from 6 to 24, and R¹ is a C₅₀₋₂ₚ alkyl optionally substituted by at least one substituent selected from the group consisting of OH, OR, N(R)₂, CₙF₂ₙ₋₁, and deuterium (D).

8. The nanoparticle of claim 3, wherein the hypoxia sensitive ligand of formula I is

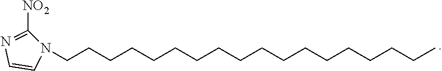 [SMILES: CCCCCCCCCCCCCCCCCCn1ccnc1[N+](=O)[O-]]

9. The nanoparticle of claim 1, wherein in the hypoxia sensitive ligand of formula II, AA is selected from the group consisting of alanine, arginine, asparagine, aspartic, cysteine, glutamine, glutamic acid, glycine, histidine, isoleucine, leucine, lysine, methionine, phenylalanine, proline, serine, threonine, tryptophan, tyrosine, and valine.

10. The nanoparticle of claim 9, wherein in the hypoxia sensitive ligand of formula II, p is 1 and (AA)ₚ is selected from the group consisting of vancomycin, daptomycin, polymix B, volcosporin, pasireotide, dalbavancin, ziconotide, oritavancin, setmelanotide, vasopressin, terlipressin, oxytocin, and cyclosporin.

11. The nanoparticle of claim 10, wherein the hypoxia sensitive ligand of formula II has the structure 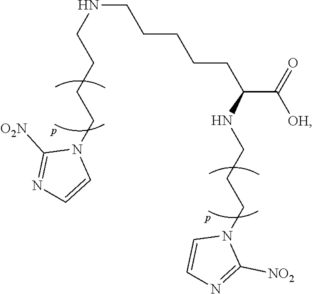
wherein each occurrence of p is independently an integer from 2 to 24.

12. The nanoparticle of claim 1, wherein the hypoxia sensitive ligand of formula III has the structure 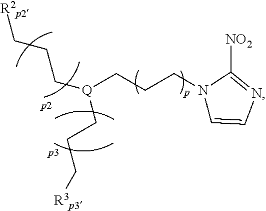
wherein p is an integer from 1 to 24, p2 is an integer from 1 to 24, p3 is an integer from 1 to 24, p2′ is an integer from 0 to 23, p3′ is an integer from 0 to 23, and R²_{p2′} and R³_{p3′} are each independently optionally substituted by at least one group selected from the group consisting of OH, OR, N(R)₂, CₙF₂ₙ₋₁, and D.

13. The nanoparticle of claim 1, wherein in the hypoxia sensitive ligand of formula V, the dendrimer is selected from the group consisting of a polyamideamine (PAMAM) dendrimer, a polypropylamine (POPAM) dendrimer, and a PAMAM-POPAM dendrimer.

14. The nanoparticle of claim 1, wherein the hypoxia sensitive ligand of formula V has the structure 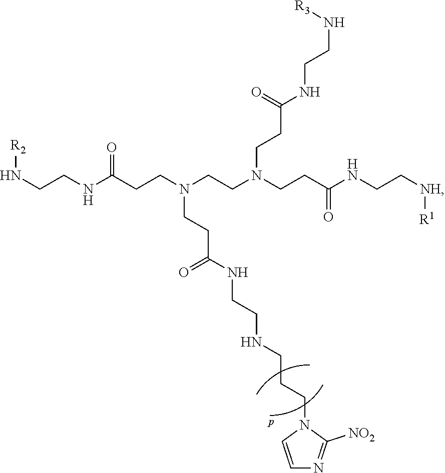
wherein p is an integer from 1 to 20, and wherein R¹, R², and R³ are each independently C₁₋₂₅ alkyl optionally substituted by at least one substituent selected from the group consisting of OH, OR, N(R)₂, CₙF₂ₙ₋₁, and D.

15. The nanoparticle of claim 1, wherein the at least one lipid, or an enantiomer or pharmaceutically acceptable salt thereof, is at least one selected from the group consisting of cholesterol, hydrogenated soy L-α-phosphatidylcholine (HSPC), 1,2-dipalmitoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 2-Oleoyl-1-palmitoyl-sn-glycero-3-phosphocholine (POPC), 1,2-distearoyl-sn-glycero-3-phosphocholine (DSPC), 1,2-dilauroyl-sn-glycero-3-phosphorylcholine (DLPC), poly(2-methacryloyloxyethyl phosphorylcholine) (PMPC), 1-O-stearoyl-2-O-oleoyl-sn-glycero-3-phosphocholine (SOPC), 1,2-dimyristoyl-sn-glycero-3-phospho-rac-(1-glycerol) (DMPG), 1,2-dilauroyl-sn-glycero-3-phospho-(1′-rac-glycerol) (DLPG), 1,3-dipalmitoyl-sn-glycero-3-phosphoethanolamine (DPPE), 1,2-dimyristoyl-sn-glycero-3-phospho-ethanolamine (DMPE), 1,2-dioleoyl-sn-glycero-3-phosphoethanolamine (DOPE), 1,2-distearoyl-sn-glycero-3-phosphoethanolamine (DSPE), 1,2-distearoyl-sn-glycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-Dimyristoyl-sn-glycero-3-phosphate (DMPA), 1,2-Dipalmitoyl-sn-glycero-3-phosphate (DPPA), 1,2-Distearoyl-sn-glycero-3-phosphate (DSPA), and PEGylated derivatives thereof comprising from 2 to 100 PEG units.

16. The nanoparticle of claim 1, wherein at least one the following applies:
(a) the at least one lipid is at least one pH sensitive lipid;
(b) the at least one lipid is independently present in an amount of about 0.5 to about 95 mol %;
(c) the at least one hydrophilic therapeutic agent is present in an amount of about 0.025 to about 95 mol %.

17. The nanoparticle of claim 1, wherein the at least one hydrophilic therapeutic agent is at least one selected from the group consisting of metformin, cerebroprotectants, immunosuppressants, immunomodulators, PPAR-γ agonists, antioxidants, alkylating agents, chemotherapeutic agents, anti-inflammatory agents, anti-apoptotic agents, sulfonylureas, cerebral vasodilators, neuroprotective peptides, angiogenic growth factors, neurogenic growth factors, oligonucleotides, nucleic acids, agonists of glycolysis, modulators/antagonists of glycolysis, lactate transporter antagonists, alkaloids, antibiotics, tyrosine kinase inhibitors, and combinations thereof.

18. The nanoparticle of claim 1, wherein the outer region further comprises at least one hydrophobic therapeutic agent,
optionally wherein at least one applies:
(a) the at least one hydrophobic therapeutic agent is at least one selected from the group consisting of pioglitazone, hydrophobic fluorescent dyes, lipids, sterols, chemotherapeutic agents, and combinations thereof;
(b) the at hydrophobic therapeutic agent is independently present in an amount of about 0.5 to about 95 mol %.

19. The nanoparticle of claim 1, wherein the nanoparticle comprises at least two hydrophilic therapeutic agents or at least one hydrophilic therapeutic agent and at least one hydrophobic therapeutic agent chosen from PPARγ agonists, PPARα agonists, steroidal anti-inflammatory drug, non-steroidal anti-inflammatory drug, thiazolidinediones, sulfonylureas, statins, biguanides, antiapoptotic agents, antioxidants, rho-associated protein kinases, and poly-ADP ribose polymerase (PARP) inhibitors.

20. The nanoparticle of claim 1, wherein at least one applies:
(a) the at least one hydrophilic therapeutic agent is encapsulated in a cyclodextrin;
(b) the at least one hydrophilic therapeutic agent is at least one selected from the group consisting of pioglitazone, metformin, uric acid, fasudil, glyburide, glipizide, fingolimod, Vitamin E, veliparib, olaparib, rucaparib, 3-aminobenzamide, pamiparib, talazoparib, lovastatin, simvastatin, and combinations thereof.

21. The nanoparticle of claim 1, further comprising a contrast agent,
optionally wherein the contrast agent is at least one selected from the group consisting of a transition metal-containing contrast agent, iron oxide-containing contrast agent, iodinated CT agents, PET radioisotopes, radioactive agents, fluorophores, quantum dots, and chemiluminescent agents,
optionally wherein the contrast agent is covalently linked to the hypoxia sensitive ligand of formula I, formula II, formula III, formula IV, or formula V.

22. The nanoparticle of claim 1, wherein the inner core further comprises a second hydrophilic therapeutic agent,
optionally wherein at least one applies:
(a) the second hydrophilic therapeutic agent enhances the permeability of the blood-brain barrier (BBB) to the nanoparticle;
(b) the second hydrophilic therapeutic agent is an A2A adenosine receptor agonist;
(c) the second hydrophilic therapeutic agent is regadenoson.

23. The nanoparticle of claim 1, further comprising a brain efflux suppressing agent, optionally wherein the brain efflux suppressing agent is a P-glycoprotein inhibitor.

24. A method of treating an ischemic or hypoxic condition in a subject, the method comprising administering to the subject in need thereof a therapeutically effective amount of the nanoparticle of claim 1.

25. The method of claim 24, wherein the ischemic or hypoxic condition is as a result of a condition selected from the group consisting of systemic ischemia, ischemic stroke, transient ischemic stroke, traumatic brain injury, organ ischemia, chemically-induced ischemia, spinal cord injury, brain contusion, concussion, and solid tumor.

26. The method of claim 25, wherein the solid tumor is at least one tissue or organ selected from the group consisting of brain, head, neck, liver, spleen, kidney, lung, skin, pancreas, breast, cervical, testicular, ovarian, eye, oral, rectum, bladder, prostate, stomach, and colon.

27. The method of claim 24, wherein at least one applies:
(a) the administration is by a route of administration selected from the group consisting of intravenous (IV), intraarterial, intraperitoneal, subcutaneous, intradermal, retroorbital, direct injection, convection enhanced delivery, intrathecal, intranasal, inhalers, sublingual, and oral administration;
(b) the administration is a bolus infusion or a continuous infusion;
(c) the subject is further administered an additional therapeutic agent, optionally wherein the additional therapeutic agent is administered concurrently or sequentially with the nanoparticle.
